(19) United States
(12) Patent Application Publication
(10) Pub. No.: US 2024/0150392 A1
(43) Pub. Date: May 9, 2024

(54) DNA GRIDIRON COMPOSITIONS AND METHODS

(71) Applicant: ARIZONA BOARD OF REGENTS ON BEHALF OF ARIZONA STATE UNIVERSITY, Scottsdale, AZ (US)

(72) Inventors: Dongran HAN, Cambridge, MA (US); Hao YAN, Chandler, AZ (US)

(21) Appl. No.: 18/228,604

(22) Filed: Jul. 31, 2023

Related U.S. Application Data
(63) Continuation of application No. 16/938,474, filed on Jul. 24, 2020, now Pat. No. 11,760,774.
Application No. 16/938,474 is a continuation of application No. 16/202,841, filed on Nov. 28, 2018, now Pat. No. 10,774,107.
Application No. 16/202,841 is a continuation of application No. 15/121,007, filed on Aug. 23, 2016, now Pat. No. 10,189,874.
(60) Provisional application No. 61/944,677, filed on Feb. 26, 2014.

(51) Int. Cl. C07H 21/04; C07H 1/00

(52) U.S. Cl. CPC C07H 21/04; C07H 1/00

(57) ABSTRACT

Novel compositions and methods for engineering wireframe architectures and scaffolds of increasing complexity by creating gridiron-like DNA structures (FIG. 1). A series of four-arm junctions are used as vertices within a network of double-helical DNA fragments. Deliberate distortion of the junctions from their most relaxed conformations ensures that a scaffold strand can traverse through individual vertices in multiple directions. DNA gridirons, ranging from two-dimensional arrays with reconfigurability to multilayer and three-dimensional structures and curved objects, can be assembled according the methods presented herein.

### CROSS-REFERENCE TO RELATED APPLICATIONS

This application is a continuation of U.S. patent application Ser. No. 16/938,474, filed on Jul. 24, 2020, which was a continuation of U.S. patent application Ser. No. 16/202,841, filed on Nov. 28, 2018, which is a continuation of U.S. patent application Ser. No. 15/121,007, filed on Aug. 23, 2016, which is a national stage entry of International Patent Application No. PCT/US2015/017553, filed on Feb. 25, 2015, which claims priority to U.S. Provisional Patent Application No. 61/944,677 filed on Feb. 26, 2014.

### STATEMENT OF GOVERNMENT RIGHTS

This invention was made with government support under N000140911118 awarded by the Office of Naval Research, 1104373 awarded by the National Science Foundation, and W911NF-11-1-0137 awarded by the Army Research Office. The government has certain rights in the invention.

### SEQUENCE LISTING

The instant application contains a Sequence Listing which has been submitted electronically in XML file format and is hereby incorporated by reference in its entirety. Said XML copy, created on Jun. 16, 2023, is named G8118-00305_SL.xml and is 362,165 bytes in size.

### FIELD OF THE INVENTION

This disclosure relates to the field of nanotechnology and more particularly to the engineering of wireframe architectures and scaffolds using DNA structures.

### BACKGROUND OF THE INVENTION

Self-assembling nucleic acid molecules have shown merit as versatile materials for organizing and constructing complex nano-scale structures. Methods are known for generation of complex DNA origami nanostructures with addressable surface features. For example, a long scaffold strand, most often the 7429-nucleotide (nt) circular genome of the M13mp18 bacteriophage, is organized and folded by interactions with a large number of short, synthetic, staple strands. The path of the scaffold strand in this approach has been restricted to discrete domains of parallel lines because it is based on the double crossover unit motif to link adjacent helices.

Because engineering wireframe architectures and scaffolds of increasing complexity is an important challenge in nanotechology, methods and compositions for achieving same are very useful and inventive.

### SUMMARY OF THE INVENTION

We present a design strategy that uses an unusual set of immobile Holliday junction analogs (four-arm junctions) as the basic structural unit of DNA origami nanostructures and as joints to construct a variety of two-dimensional (2D) and 3D gridiron structures, in which the scaffold strand and corresponding double helices are not restricted to a 1D parallel, raster-fill pattern. By programming the connection between individual joints with DNA segments of variable lengths, we constructed complex wireframe geometries.

These and other aspects of the invention will be apparent upon reference to the following detailed description and figures. To that end, any patent and other documents cited herein are hereby incorporated by reference in their entirety.

### BRIEF DESCRIPTION OF THE FIGURES

The patent application file contains at least one drawing executed in color. Copies of this patent or patent application publication with color drawing(s) will be provided by the Office upon request and payment of the necessary fee.

FIG. 1. (A) (Left) Geometry and strand polarity of a single gridiron unit formed from four four-arm junctions. (Right) Geometry and polarity of a double-crossover molecule motif used in conventional DNA origami structures. For both structures, the ssDNAs depicted in red are components of DNA double helices that serve as the scaffold strands. The ssDNA depicted in gray represents staple strands. (B) Models of four four-arm junction molecules in their relaxed conformation. The orientation of the upper two junctions differs from that of the lower two by a 180° in-plane rotation. Thus, the polarities of the continuous red strands in the upper and lower layers of the horizontally oriented helices are antiparallel to one another. (C) Models illustrating the deviation from a relaxed conformation required of the four individual junctions to form a gridiron unit. The blue arrows indicate that the top helix of the junctions in the upper-left and lower-right corners must be rotated ˜150° clockwise, whereas in the upper-right and lower-left junctions they must be rotated ˜30° counterclockwise. (D and E) Helical models illustrating a complete gridiron unit. (F and G) Schematics illustrating a typical scaffold-folding path for a 2D DNA gridiron pattern.

FIG. 2. (A to D) Images for a 2D gridiron structures with 21-bp cavities with AFM [(A) and (B)] and TEM images [(C) and (D)]. (E and F) Images for a 2D gridiron with 63-bp cavities with AFM (E) and TEM images (F). (G to J) Schematics (left), TEM images (middle), and histogram analysis (right) of the angle distributions for angle control. All scale bars indicate 200 nm, and all zoom-in images (images without scale bars) are 200 by 200 nm.

FIG. 3. Multilayer gridiron design strategies. (A and B) Strategy 1 is stacked layers. (A) A portion of a double-layer gridiron lattice with 52-bp cavity size. The yellow circles designate the permissible connection points to a third layer. The dashed lines correspond to possible connection points to form additional layers. (B) Given the double-layer gridiron lattice (X and Y lengths) and the distance between crossover points in the third layer, the angle q can be calculated as 180°-cos-1[(X2+Y2−L2)/2XY]. (C) Strategy 2 is intertwining gridiron planes. (D to F) Schematics (left), AFM (middle), and TEM (right) images of (D) a three-layer hexagonal gridiron design, q=120°; (E) a four-layer gridiron design, q is not controlled because the dashed green line in (A) represents a connection strategy that cannot fix the angle; and (F) a 3D gridiron assembled by using strategy 2. All scale bars indicate 200 nm, and all zoom-in images (images without scale bars) are 200 by 200 nm.

FIG. 4. Schematics (left), AFM (middle), and TEM images (right) of (A) an S-shaped structure, (B) a sphere, and (C) a screw. All scale bars indicate 200 nm, and all zoom-in images (images without scale bars) are 200 by 200 nm. In (B) and (C), the diameter and the width, respectively, appear to be larger in the AFM images compared with the TEM images. This difference is probably a result of flattening of the 3D objects into two-layer structures and AFM tip convolution.

FIG. 5 shows sequences of the staples in the 21 bps Gridiron structure.

### DETAILED DESCRIPTION OF THE PREFERRED EMBODIMENTS

Although intuitively one could imagine threading a single-stranded scaffold through a number of four-arm junction units in both horizontal and vertical directions to create gridiron like patterns, the structural properties of traditional Holliday junction impose certain challenges that require unconventional rearrangement of the junction unit conformation, as revealed by the design principles described below.

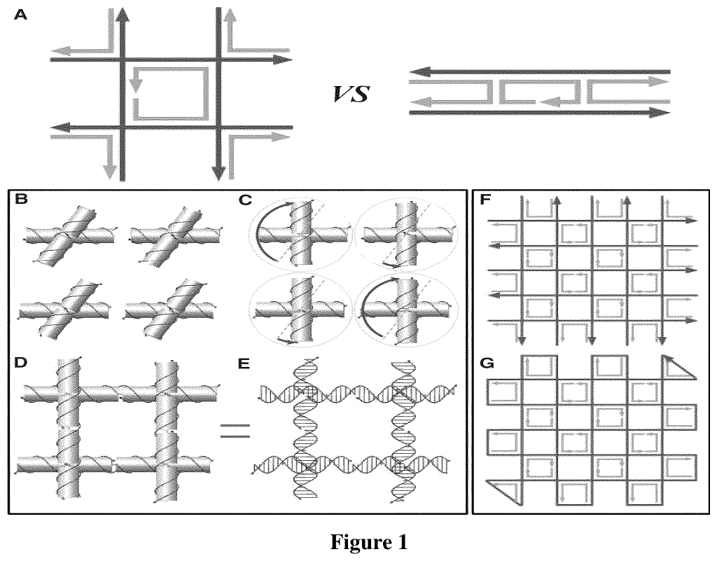

We compared a gridiron unit to a double crossover motif (FIG. 1A), and the DNA strands are abstracted to display only their polarity with the arrows pointing from 5′ to 3′. In the gridiron unit, four four-arm junctions are linked together to form a two-layer square frame in which the helices on opposite sides lie in the same plane. An antiparallel arrangement between opposite sides of the square frame permits a single, central strand to traverse each of the helices.

Each of the four junctions is depicted in its relaxed conformation (FIG. 1B) such that the helices form a right-handed twist with a 60° torsion angle. Deviation from a relaxed conformation is required of each junction to form the gridiron unit cell. First, the red strands in the horizontally oriented helices (both top and bottom images) can be linked together to produce continuous strands without reversing the 5?-to-3? polarity (FIGS. 1, B and C). Next, the vertically oriented helices need to be rotated in the plane about the junction points (FIG. 1C) to allow the formation of continuous 5′-to-3′ connections between upper and lower junctions (FIGS. 1, D and E).

Connecting a number of gridiron units leads to the formation of a variety of 21) lattices (FIGS. 1, F and G). The red lines represent the DNA strands that are expected to retain an unperturbed helical structure with continuous base stacking. Meanwhile, the short strands (in gray) form the crossovers between helical domains and function as staples. A long scaffold strand is created by connecting the termini of the red strands with short single-stranded DNA (ssDNA) loops. In the most basic design, the scaffold begins at one corner, fills the first layer, changes direction at the opposite corner, and then fills the second layer to produce a structure in which the helices within the two layers are oriented perpendicularly with respect to each other. Lastly, the scaffold returns to its initial position to form a closed loop (FIG. 1G).

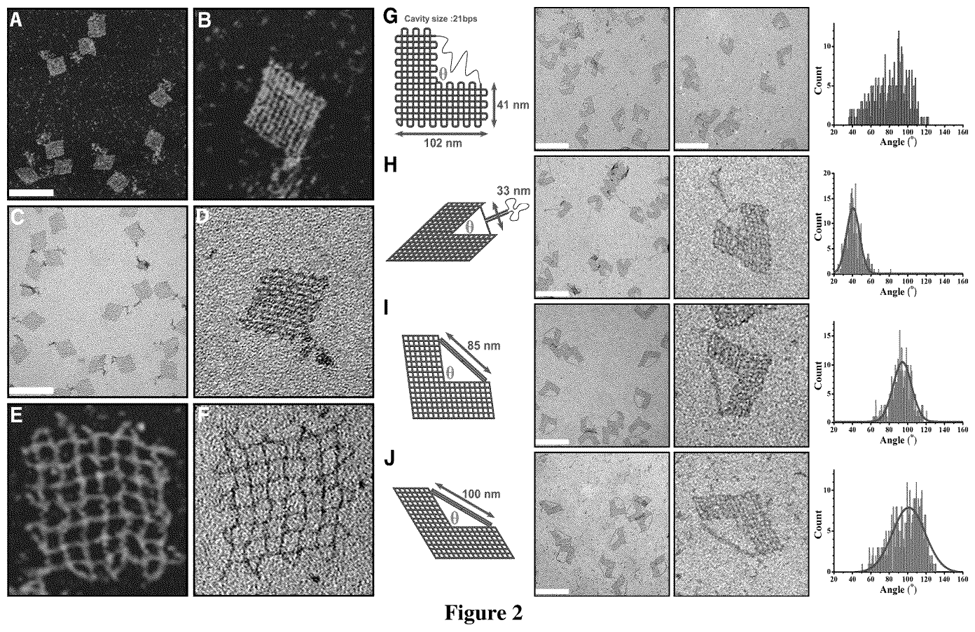

The cavity size of gridiron structures can be tailored by altering the number of base pairs between the adjacent junction points. An 1.1-by-1.1 gridiron structure (11 vertical helices by 11 horizontal helices) with 21 base pairs (bp) between junctions in both directions uses 5301 of 7249 nt of the M13mp18 ssDNA scaffold strand and contains 120 staple strands (42 nt each). The remaining 1948 nt of the scaffold form a single-stranded loop at one corner that is visible in atomic force microscope (AFM, FIGS. 2, A and B) and transmission electron microscope (TEM) images (FIGS. 2, C and D). Gridiron structures with 63-by-63-bp cavities (FIGS. 2, E and F) were assembled to demonstrate the programmability of the design strategy.

To test whether the ssDNA scaffold is required to force the junction to rotate and form the intended gridiron structures, we designed and successfully constructed a scaffold-free 11-by-11 gridiron structure. We also found that scaffolded and scaffold-free gridiron elements can be combined within a single structure. Further, a scaffold-free gridiron unit was examined h native gel electrophoresis to verify its formation when the component strands were mixed in equal stoichiometric ratios. Although the schematic diagram in FIG. 1D depicts 90° angles between the helices in the upper and lower layers, the angles are not fixed because the junctions are flexible. The experimental results reveal the formation of rhomboid rather than square structures; the junctions most likely behave cooperatively in order to maintain optimized base-stacking interactions and the lowest overall free energy. The single-stranded scaffold loop in one corner serves as an intrinsic marker to determine the angles adopted by the gridiron, and the angles display a bimodal distribution with nearly equal amplitudes, centered at 76° T 7° (SD) and 103° T 7°.

The flexibility of the joints makes it possible to control or reconfigure the conformation of the gridiron structure by exerting external forces on selected corners of a gridiron. A modified version of a 15-by-15 gridiron structure with 21-bp cavities has about one quadrant of the gridiron unfolded and forms a randomly coiled 836-nt single-stranded loop between two “arms” of tweezers (FIG. 20). The ssDNA loop is long enough to allow the structure to adopt a relaxed conformation. The observed distribution of the inner angle (q) of the tweezers (measured from 309 individual structures) is broad and centered at 80° to 90°.

We could contract and extend the ssDNA loop by introducing secondary or tertiary structures that generate enough force to control the angle. Sets of staple strands were designed to either contract the ssDNA loop and fix an acute angle (a narrow distribution centered at 41° T 7°) via the formation of a two-helix bundle (FIG. 2H) or to extend the loop to secure a right (FIG. 2I) or obtuse angle (FIG. 2J) via the formation of a three-helix bundle of specific length. The design with the right angle shows a narrow and symmetrical distribution centered at 94° T 10°, and the design with the obtuse angle has a broader angle distribution centered at 102° and exhibits an asymmetry that is more heavily weighted toward smaller angles.

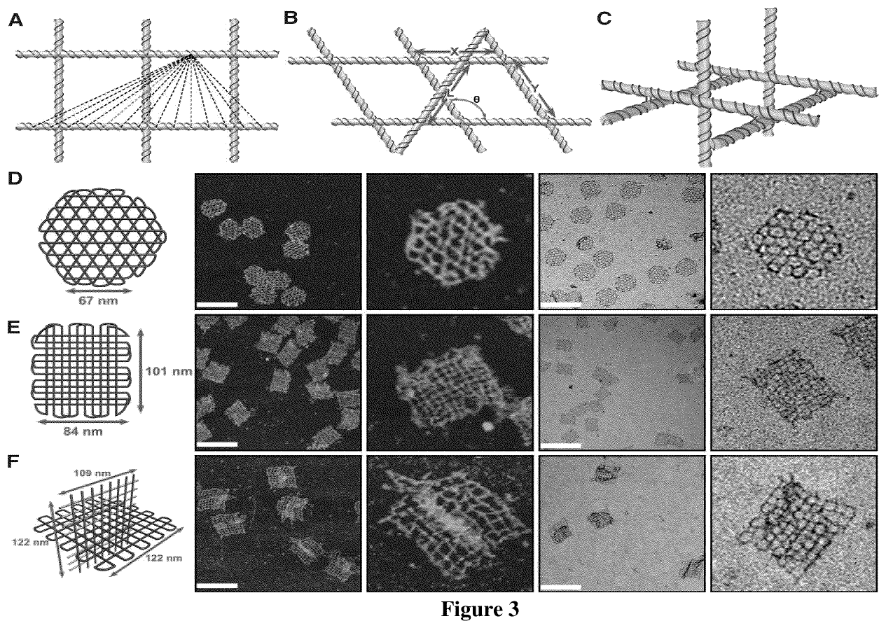

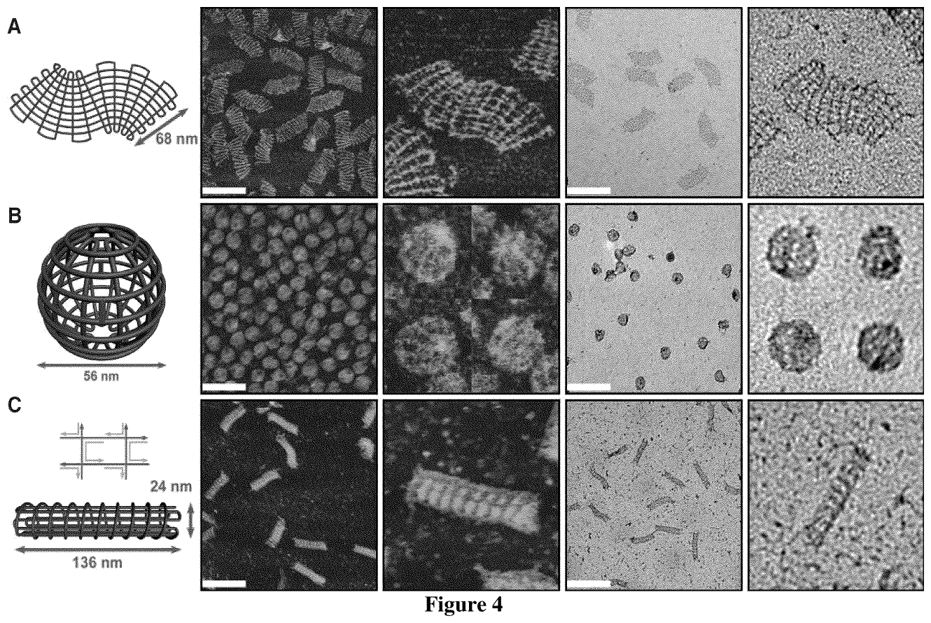

We extended the gridiron design into the third dimension by three different strategies. The first involves stacking multiple layers of 2D gridiron lattices at selected connection points (FIGS. 3, A and B). The second relies on intertwining several gridiron planes in x-y-z directions (FIG. 3C). The third method has its basis in distorting a single layer of DNA gridiron into 3D structures by controlling their curvatures (FIG. 4). By using the first strategy, we constructed a three-layer hexagonal (FIG. 3D), a four-layer rectangular gridiron (FIG. 3E), and a three-layer parallelogram structure. For all multilayer gridiron structures, the scaffold strand raster fills each layer, with an off-set in the angle formed between the helices of adjacent layers. The three-layer hexagonal and four-layer rectangular structures maintained 60° and 90° offsets between layers, respectively.

Varying the location and distance between connection points will yield differently patterned multilayer structures. In contrast to the angle flexibility present in the quasi-2D structures, the addition of a third layer fixes the angles at junction points. The only exception to this is for connections through the center of the same unit motif, as shown by the green dashed line (FIG. 3A). In a 3D model of an eight-by-eight-by-eight three-layer hexagonal gridiron structure (FIG. 3D), neighboring junctions in the top and bottom layers are 52 by apart, and neighboring junctions in the middle layer (alternating connections to the top and bottom layers) are 2.6 bp apart. Because X=I=L (FIG. 3B), each junction should adopt a 60° torsion angle. A four-laver rectangular gridiron structure (FIG. 3E) can be broken down into two six-by-five double-layer gridirons (with 52-bp cavities) stacked on top of one another with a 26-bp offset in the connections between the first and third, and second and fourth, layers.

The relations of the lattice planes in gridiron structures are not restricted to stacked multilayer structures. The 3D gridiron structures can also be assembled by integrating gridiron lattices with scaffold-free elements. FIG. 3F presents such a design in which a nine-by-nine gridiron plane (shown in blue) is intertwined with an eight-by-eight scaffold-free gridiron plane (shown in yellow). The complex, interwoven topology of this particular structure required combining scaffolded and scaffold-free components.

Gridiron designs can allow assembly of even more complex structures by inducing a desired curvature in the basic structural unit described in nonparallel helices. The relation between adjacent linear helices (the angles formed by their theoretical intersection) between adjacent linear helices was varied. Some 3D gridiron structures that contain curvature were also achieved, such as the sphere shown in FIG. 4B. The helices in concentric ring and radial spoke layers are stretched in the center and shrunk at the edges, forming a latitudinal and longitudinal framework, respectively. This is realized by progressively adjusting the distance between junctions in latitudinal directions. Additional modifications to the basic structural motif can be used to produce other complex structures. In the screw structure (FIG. 4C), the polarity of the DNA strands in the square unit motif differs from what is illustrated in FIG. 1B (where adjacent scaffold helices have an antiparallel polarity in one direction and the same polarity in the other direction). The scaffold strand is arranged in an antiparallel configuration to form is wireframe cylinder structure (11 helices are arranged axially) and subsequently wraps around the cylinder (analogous to a left-handed screw) until the two ends meet. The distance between adjacent axial helices is 21 bp, the interthread distance is 42 bp, and the AFM and TEM images display the expected left-handed conformation.

The design principles of creating gridiron units allow scaffold strands to travel in multiple directions, which represent an important departure from certain aspects of the previous DNA origami methods. Traditional Holliday junctions do not naturally adopt conformations that would allow them to be connected in such a way, and it was unexpected to find that these motifs could (within a larger network of crossovers) endure a 150° rotation of one of the arms while simultaneously maintaining their integrity. Indeed, the flexible and dynamic behavior of these motifs may have excluded these types of junction conformations for consideration in scaffolded structures. Yield analysis from agarose gel and TEM images shows that the structures, even without purification, form with reasonably high yield (from ˜36% for the gridiron tweezers to ˜85% for the gridiron screw, estimated from agarose gels; from ˜51% for the gridiron sphere to ˜89% for the four-layer gridiron, estimated from TEM images; see supplementary materials for yield analysis). The ability to engineer DNA gridirons that are analogous to vector-based objects, where a series of points with defined positions in 3D space are connected by lines, is an important milestone in the development of synthetic nucleic acid structures. In particular, this opens up new opportunities to implement the design of complex wireframe structures that are amenable to dynamic controls. A future challenge in DNA origami is to achieve true folding, starting from a 2D sheet (miura ori), rather than the 1D M13 scaffolds commonly used in traditional DNA origami construction. The loose 2D networks and freely rotating hinges between different planes of DNA gridirons provide the design features necessary to implement Miura ori type of origami.

### EXAMPLES

It should be understood that these Examples, while indicating preferred embodiments of the invention, are given by way of illustration only. From the above discussion and these following Examples, one skilled in the art can ascertain the essential characteristics of this invention, and without departing from the spirit and scope thereof, can make various changes and modifications of the invention to adapt it to various uses and conditions.

### Materials and Methods

All staple strands were purchased from Integrated DNA Technologies Inc. (www.IDTDNA.com) in the format of 96-well plates at a 25 nmole synthesis scale. All strands were normalized to 200 μM×100 μL and were used without further purification. M1.3mp18 single stranded DNA was purchased from New England Biolabs (NEB, Catalog number: #N4040S) and was used as received.

Assembly of 2D and 3D DNA nanostructures. The design and sequences of the DNA oligos used to form a particular structure are listed below. For each design, 10 nM of single stranded. M13mp18 DNA (7,249 nucleotides) was mixed with a 10 times molar excess of staple strands in TAE Mg2+ buffer (40 mM Tris, 20 mM Acetic acid, 2 mM EDTA and 12.5 mM Magnesium acetate, pH 8.0). The resulting solutions were annealed from 95° C. to 4° C. to form the designed structures. The exact temperature steps for the slow anneal are as follows: 94 to 86° C. at 4° C. per 5 minutes; 85 to 70° C. at 1° C. per 5 minutes; 70 to 40° C. at 1° C. per 15 minutes: 40 to 25° C. at 1° C. per 10 minutes. The exact temperature steps for the fast anneal are as follows: 90 to 76° C. at 2° C. per 5 minutes; 76 to 24° C. at 4° C. per 5 minutes. All structures form in both anneal protocols. All samples are then subjected to A imaging and TEM imaging without further purification.

AFM imaging. For AFM imaging, the sample (2 L) was deposited onto a freshly cleaved mica surface (Ted Pella, Inc.) and left to adsorb for 2 min 50 L buffer (1×TAE-Mg2+, plus 2 L 1.00 mM NiCl2) was added onto the mica, and the sample was scanned on a Veeco 8 AFM in the Scanasyst in Fluid mode using scanasyst in fluid+tips (Veeco, Inc.).

TEM imaging: TEM samples were prepared by dropping 2 μL of the sample solution on a carbon-coated grid (400 mesh, Ted Pella). Before depositing the sample, the grids were negatively glow discharged (Emitech K100X). After 1 minute, the excess sample was wicked away from the grid with a piece of filter paper. To remove the excess salt, the grid was washed with a drop of nanopure water and the excess water was wicked away with filter paper. For staining, the grid was treated with a drop of 0.7% uranyl formate solution and the excess solution was removed with filter paper. The grid was treated with a second drop of uranyl formate solution for 20 seconds, and the excess solution was removed with filter paper. The grid was subsequently held at room temperature in air to evaporate the excess solution. TEM studies were conducted with a Philips CM12 transmission electron microscope, operated at 80 kV in bright field mode.

Agarose Gel electrophoresis: The folding products were subject to native gel electrophoresis on 0.75% agarose gel (1×TAE-Mg2+, preloaded in the gel with 0.5 μg/mL ethidium bromide) at 75-80 V for two to three hours and the gels were visualized under UV light.

Page Gel electrophoresis: The folding products were subject to native gel electrophoresis on 6% Native PAGE gel (polyacrymide; 1×TAE-Mg2+) at 200V for 2 hours at 20 degree and the gels were visualized under UV light.

Design details and sequences of assembled structures. “Tiamat” software was used to design all DNA Gridiron structures. Tiamat is a basic DNA drawing software program (similar programs also exist) and no special algorithms were used to design the DNA Gridiron structures. Most of the design tasks were performed manually and Tiamat was primarily used to generate staple strands sequences according to the scaffold strand sequence.

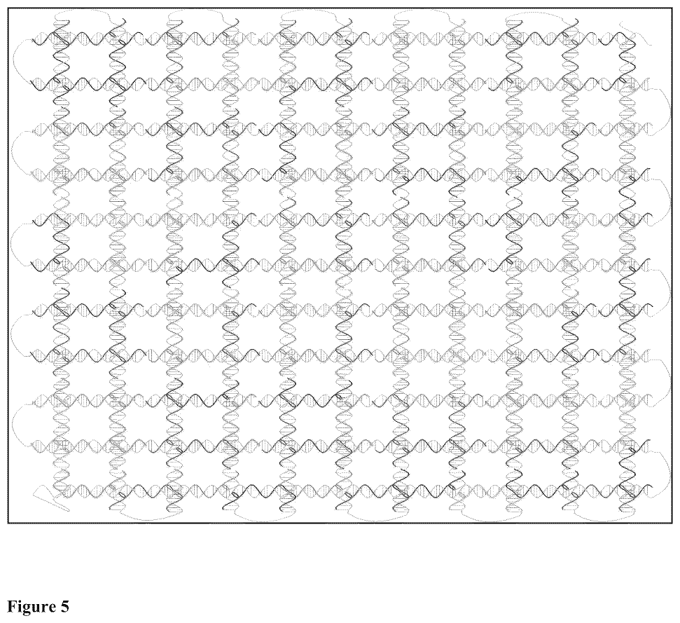

FIG. 5 illustrates the design details and staple strand sequences of some example DNA Gridiron structures. Tiamat software and files for all designs are available for downloading at the following website: skydrivedive.com/redir?resid=2416F4B1C095AF65!152&authkey=!AELrUerdPdo1P1w.

TABLE 1
Sequences of the staples in the 21 bps Gridiron structure

| Name (No.) | Sequence |
|---|---|
| 21bpsGridiron-1 | GAAAATTCATAAGTAAGCGTCATACATGGCTTAGACGGGAGA |
| | (SEQ ID NO. 1) |
| 21bpsGridiron-2 | ATATTCACAAAATAAAAACAGGGAAGCGCATTTTGATGATAC |
| | (SEQ ID NO. 2) |
| 21bpsGridiron-3 | AGGAGTGTACTAATAAACAGCCATATTATTTAATTGGCCTTG |
| | (SEQ ID NO. 3) |
| 21bpsGridiron-4 | GCCAGTTACAAGGTAATAAGTTTTAACGGGGTGTCCTGAACA |
| | (SEQ ID NO. 4) |
| 21bpsGridiron-5 | GCAGAACGCGCGAGGTTGAGGCAGGTCAGACGTCCCAATCCA |
| | (SEQ ID NO. 5) |
| 21bpsGridiron-6 | AATAAGAAACGGACTTGAGCCATTTGGGAATTTTCAGCTAAT |
| | (SEQ ID NO. 6) |
| 21bpsGridiron-7 | GAGAGAATAACCAAATAAATCCTCATTAAAGCAAAAGGGCGA |
| | (SEQ ID NO. 7) |
| 21bpsGridiron-8 | AGCATTGACAGCTGTTTATCAACAATAGATAACAGTGCCTTG |
| | (SEQ ID NO. 8) |
| 21bpsGridiron-9 | AGTAACAGTGCCCAGTAATAAGAGAATATAAAAGCCGCCGCC |
| | (SEQ ID NO. 9) |
| 21bpsGridiron-10 | GCATTTTCGAGCCGTATAAACAGTTAATGCCCTATCAAAATC |
| | (SEQ ID NO. 10) |
| 21bpsGridiron-11 | ATTAAGACGCTCGCCACCAGAACCACCACCAGGTACCGACAA |
| | (SEQ ID NO. 11) |
| 21bpsGridiron-12 | AAGGTAAAGTAAGCACCATTACCATTAGCAAGGATAGCTTAG |
| | (SEQ ID NO. 12) |
| 21bpsGridiron-13 | TTTACCGTTCCTGGTTTACCAGCGCCAAAGACCAGAATGGAAAG |
| | (SEQ ID NO. 13) |
| 21bpsGridiron-14 | CATTCAACCGATTGACGGAAATTATTCATTAAAGCCTTTACA |
| | (SEQ ID NO. 14) |
| 21bpsGridiron-15 | GAAAATAGCAGGTGAATTATCACCGTCACCATTTTTTGTT |
| | (SEQ ID NO. 15) |
| 21bpsGridiron-16 | GCAAAGACACCGTAAATGAATTTTCTGTATGGTAATTGAGCG |
| | (SEQ ID NO. 16) |
| 21bpsGridiron-17 | TAGCATTCCACACCCTGAACAAAGTCAGAGGGGATTTTGCTA |
| | (SEQ ID NO. 17) |
| 21bpsGridiron-18 | AACAACTTTCACGCTAACGAGCGTCTTTCCAGACAACGCCTG |
| | (SEQ ID NO. 18) |
| 21bpsGridiron-19 | AATCTTACCAAACAGTTTCAGCGGAGTGAGAAATGTAGAAAC |
| | (SEQ ID NO. 19) |
| 21bpsGridiron-20 | AGAAAAATAATGTTTCGTCACCAGTACAAACTAGCCTAATTT |
| | (SEQ ID NO. 20) |
| 21bpsGridiron-21 | ATTAACTGAACAGACAGCCCTCATAGTTAGCGACAATCAATA |
| | (SEQ ID NO. 21) |
| 21bpsGridiron-22 | AACAACATGAGAGCCAGCAAAATCACCAGTATTCTGTCCA |
| | (SEQ ID NO. 22) |
| 21bpsGridiron-23 | CGTAACACTGAATCCCATCCTAATTTACGAGCTAGAAAGGAA |
| | (SEQ ID NO. 23) |
| 21bpsGridiron-24 | CAACTAAAGGATTAACAACGCCAACATGTAATACCCATGTAC |
| | (SEQ ID NO. 24) |
| 21bpsGridiron-25 | AATCGCCATATATTGCGAATAATAATTTTTTCGCTTAGGTTG |
| | (SEQ ID NO. 25) |
| 21bpsGridiron-26 | ATAGGTCTGAGGGGATAGCAAGCCCAATAGGATTAGGCAGAG |
| | (SEQ ID NO. 26) |
| 21bpsGridiron-27 | TCCAGACGTTAACGGAATAAGTTTATTTTGTCTAACGATCTAAA |
| | (SEQ ID NO. 27) |
| 21bpsGridiron-28 | TCGCCCACGCAGCCATTGCAACAGGAAAAATGCGCCGACA |
| | (SEQ ID NO. 28) |
| 21bpsGridiron-29 | CCTCATTTTCAAGACTACCTTTTTAACCTCCGACGTTGAAAA |
| | (SEQ ID NO. 29) |
| 21bpsGridiron-30 | TCTCCAAAAAATGAATTACCTTTTTTAATGGAGAGCCACCAC |
| | (SEQ ID NO. 30) |
| 21bpsGridiron-31 | ATATAAGTATTTGACGCTCAATCGTCTGAAGATAAGTGCC |
| | (SEQ ID NO. 31) |
| 21bpsGridiron-32 | ACCCTCAGAGCGAGAAGAGTCAATAGTGAATTCCTGCCTATT |
| | (SEQ ID NO. 32) |
| 21bpsGridiron-33 | TCGGAACCTATTGTGAGTGAATAACCTTGCTTCAGAGCCACC |
| | (SEQ ID NO. 33) |
| 21bpsGridiron-34 | CGTTTGCCAATTCACCAGTCACACGACCAGTTCGGTCATA |
| | (SEQ ID NO. 34) |
| 21bpsGridiron-35 | AAACATAGCGCCGGAAACGTCACCAATGAATAATTTTCCC |
| | (SEQ ID NO. 35) |
| 21bpsGridiron-36 | ACAATTTCATTAAGGCTCCAAAAGGAGCCTTTTATACTTCTG |
| | (SEQ ID NO. 36) |
| 21bpsGridiron-37 | GAACAAAGAATCAGTAGCGACAGAATCAAGTTGAGTAACA |
| | (SEQ ID NO. 37) |
| 21bpsGridiron-38 | GATAATACATTTGCTTTCGAGGTGAATTTCTTAGCCCTAAAA |
| | (SEQ ID NO. 38) |
| 21bpsGridiron-39 | ACAGAGATATAGCGCGTTTTCATCGGCATTTAATAAAAGG |
| | (SEQ ID NO. 39) |
| 21bpsGridiron-40 | ATTTTAAAAGTTTTGCCTTTAGCGTCAGACTGGAACCCTTCT |
| | (SEQ ID NO. 40) |
| 21bpsGridiron-41 | GACCTGAAAGCCGGAACCAGAGCCACCACCGGAACGTTATTA |
| | (SEQ ID NO. 41) |
| 21bpsGridiron-42 | ATCAAAATCACGTAAGAATACGTGGCACAGACGGTTTTGCTC |
| | (SEQ ID NO. 42) |
| 21bpsGridiron-43 | AGTACCAGGCGATGGATTATTTACATTGGCAGTCTTTTCATA |
| | (SEQ ID NO. 43) |
| 21bpsGridiron-44 | AATTCGACAACGAGAAGGATTAGGATTAGCGGAATATTTTTG |
| | (SEQ ID NO. 44) |
| 21bpsGridiron-45 | AATGGCTATTACCGTACTCAGGAGGTTTAGTACTTTACAAAC |
| | (SEQ ID NO. 45) |
| 21bpsGridiron-46 | TAGGTGTATCAGTCTTTAATGCGCGAACTGATAAACAGCTTG |
| | (SEQ ID NO. 46) |
| 21bpsGridiron-47 | ATACCGATAGTCGCTCATGGAAATACCTACATTAGCCCGGAA |
| | (SEQ ID NO. 47) |
| 21bpsGridiron-48 | GTTTATCAGCTTGAGGATTTAGAAGTATTAGACCGCCACCCT |
| | (SEQ ID NO. 48) |
| 21bpsGridiron-49 | CAGAACCGCCATATAATCCTGATTGTTTGGATAATTGTATCG |
| | (SEQ ID NO. 49) |
| 21bpsGridiron-50 | AGACTCCTCAATCGTATTAAATCCTTTGCCCGAACCGCCTCC |
| | (SEQ ID NO. 50) |
| 21bpsGridiron-51 | CTCAGAGCCGCTATCATCATATTCCTGATTATTAAGAGGCTG |
| | (SEQ ID NO. 51) |
| 21bpsGridiron-52 | TCGCTATTAATACCATCGATAGCAGCACCGTAAACCACCAGA |
| | (SEQ ID NO. 52) |
| 21bpsGridiron-53 | AGGAGCGGAATCACCCTCAGAACCGCCACCCTCTGTAAATCG |
| | (SEQ ID NO. 53) |
| 21bpsGridiron-54 | AAATCAATATATATTCTGAAACATGAAAGTATCAGATGATGG |
| | (SEQ ID NO. 54) |
| 21bpsGridiron-55 | CAATTCATCAACCCTCAGAACCGCCACCCTCAAACAGTACAT |
| | (SEQ ID NO. 55) |
| 21bpsGridiron-56 | GGTTATATAACCGGCTACAGAGGCTTTGAGGACTTAATTGAG |
| | (SEQ ID NO. 56) |
| 21bpsGridiron-57 | TAAGGCGTTCCCAATTCTGCGAACGAGTAGTGAAATACCG |
| | (SEQ ID NO. 57) |
| 21bpsGridiron-58 | GCTTAATTGCTAACGCAATAATAACGGAATACAGGTCATTTTTG |
| | (SEQ ID NO. 58) |
| 21bpsGridiron-59 | TAATTTCATCTACTTCAAATATCGCGTTTTAATCATAATTAC |
| | (SEQ ID NO. 59) |
| 21bpsGridiron-60 | TAGAAAAAGCCGTTTACCAGACGACGATAAAAATATTTTAGT |
| | (SEQ ID NO. 60) |
| 21bpsGridiron-61 | AGTTGAGATTTAGACTCCTTATTACGCAGTATATTATTACAGGT |
| | (SEQ ID NO. 61) |
| 21bpsGridiron-62 | AAGACAAAGAATAATCATTGTGAATTACCTTATACAAATTCT |
| | (SEQ ID NO. 62) |
| 21bpsGridiron-63 | TACCAGTATAATCCATGTTACTTAGCCGGAACATCCAATCGC |
| | (SEQ ID NO. 63) |
| 21bpsGridiron-64 | ATCTTTGACCCATACATAAAGGTGGCAACATAGGCAAAAGAATA |
| | (SEQ ID NO. 64) |
| 21bpsGridiron-65 | AACCGAGGAGAATATAATGCTGTAGCTCAAGCCGAACAAA |
| | (SEQ ID NO. 65) |
| 21bpsGridiron-66 | CTAATATCAGAGCACCAACCTAAAACGAAAGATAAAAGAAAC |
| | (SEQ ID NO. 66) |
| 21bpsGridiron-67 | CCACTACGAAGGAGATAACCCACAAGAATTGAAAACAAAGTA |
| | (SEQ ID NO. 67) |
| 21bpsGridiron-68 | CAACGGAGATTCTATTTTGCACCCAGCTACAAATACGTAATG |
| | (SEQ ID NO. 68) |
| 21bpsGridiron-69 | TAGAAGGCTAAGTACGGTGTCTGGAAGTTTCCCAATAGCA |
| | (SEQ ID NO. 69) |
| 21bpsGridiron-70 | CAATCAATAATAGTTTCCATTAAACGGGTAAATTTTATCCTG |
| | (SEQ ID NO. 70) |
| 21bpsGridiron-71 | TTTCATGAGGACGGCTGTCTTTCCTTATCATTGTGTCGAAAT |
| | (SEQ ID NO. 71) |
| 21bpsGridiron-72 | CCGCGACCTGCAGCCAACGCTCAACAGTAGGGCTAAAGACTT |
| | (SEQ ID NO. 72) |
| 21bpsGridiron-73 | AAGATTAGTTGTGTATCATCGCCTGATAAATTCCAAGAACGG |
| | (SEQ ID NO. 73) |
| 21bpsGridiron-74 | GTATTAAACCAATTATACCAGTCAGGACGTTGGCCTTAAATC |
| | (SEQ ID NO. 74) |
| 21bpsGridiron-75 | AGAACTGGCTCAGTACCGCACTCATCGAGAACAGCAACACTA |
| | (SEQ ID NO. 75) |
| 21bpsGridiron-76 | TCATAACCCTCTGTTTAGTATCATATGCGTTATGCGATTTTA |
| | (SEQ ID NO. 76) |
| 21bpsGridiron-77 | AGCGAACCTCCGGAATTACGAGGCATAGTAAGAAGCAAGCCG |
| | (SEQ ID NO. 77) |
| 21bpsGridiron-78 | TTTTTATTTTCGACCGGAAGCAAACTCCAACAGAGGCGTTTT |
| | (SEQ ID NO. 78) |
| 21bpsGridiron-79 | AAAGCGAACCAATCGTAGGAATCATTACCGCGCATTCCATAT |
| | (SEQ ID NO. 79) |
| 21bpsGridiron-80 | AACAGTTGATTAAATAAGAATAAACACCGGAATTCGAGCTTC |
| | (SEQ ID NO. 80) |
| 21bpsGridiron-81 | ATATGCAACTATATCCGGTATTCTAAGAACGCGGTCAGGATT |
| | (SEQ ID NO. 81) |
| 21bpsGridiron-82 | AGAGAGTACCTTTTAAGAAAAGTAAGCAGATACATGTTTTAA |
| | (SEQ ID NO. 82) |
| 21bpsGridiron-83 | TAACGCCAAAACGACTTGCGGGAGGTTTTGAAGGAAGAAAAA |
| | (SEQ ID NO. 83) |
| 21bpsGridiron-84 | TCTACGTTAATAAGAAACAATGAAATAGCAATTGCAGATACA |
| | (SEQ ID NO. 84) |
| 21bpsGridiron-85 | GTAGAAAATACCCAGCGATTATACCAAGCGCGGTTAAGCCCA |
| | (SEQ ID NO. 85) |
| 21bpsGridiron-86 | ATAATAAGAGCAAAACGAACTAACGGAACAACGTTAGCAAAC |
| | (SEQ ID NO. 86) |
| 21bpsGridiron-87 | TGGCATGATTAAGGAATACCACATTCAACTAAAGCTATCTTA |
| | (SEQ ID NO. 87) |
| 21bpsGridiron-88 | CCGAAGCCCTTTTAATTGCTCCTTTTGATAAGCCAAAAGAAC |
| | (SEQ ID NO. 88) |
| 21bpsGridiron-89 | AAGCCCGAAAGTCTGACCTAAATTTAATGGTTATTTAGTTTG |
| | (SEQ ID NO. 89) |
| 21bpsGridiron-90 | ACCATTAGATAGATTGCTTTGAATACCAAGTTGATTAAGAGG |
| | (SEQ ID NO. 90) |
| 21bpsGridiron-91 | TAAACAGTTTTTGATTAGTAATAACATCACCATTGAATCC |
| | (SEQ ID NO. 91) |
| 21bpsGridiron-92 | AATTTCAACTTCGCGAGAAAACTTTTTCAAATACCAAAATAG |
| | (SEQ ID NO. 92) |
| 21bpsGridiron-93 | CGAGAGGCTTTTTATTCATTTCAATTACCTGAGAGATGGTTT |
| | (SEQ ID NO. 93) |
| 21bpsGridiron-94 | ATTCATTACAACTATCGGCCTTGCTGGTAAAGTAATCTTG |
| | (SEQ ID NO. 94) |
| 21bpsGridiron-95 | GAGGGTAGCAATATATGTAAATGCTGATGCAAGAGGCGCAGA |
| | (SEQ ID NO. 95) |
| 21bpsGridiron-96 | CGGTCAATCATAACATCAAGAAAACAAAATTAGCATCGGAAC |
| | (SEQ ID NO. 96) |
| 21bpsGridiron-97 | GATTCGCCTCATTTCGCAAATGGTCAATAATTACATCGGG |
| | (SEQ ID NO. 97) |
| 21bpsGridiron-98 | AATAATGGAAGCACCCTCAGCAGCGAAAGACAATTACATTTA |
| | (SEQ ID NO. 98) |
| 21bpsGridiron-99 | TGCGGGATCGTGGTTAGAACCTACCATATCAATTTGAAAGAG |
| | (SEQ ID NO. 99) |
| 21bpsGridiron-100 | GACAGATGAACACTAACAACTAATAGATTAGAAGGCCGCTTT |
| | (SEQ ID NO. 100) |
| 21bpsGridiron-101 | CTCAAATATTTGGGGCGCGAGCTGAAAAGGTCTAAAGCAT |
| | (SEQ ID NO. 101) |
| 21bpsGridiron-102 | CATCGCCATTACTGAGGCTTGCAGGGAGTTAAGCCGTCAATA |
| | (SEQ ID NO. 102) |
| 21bpsGridiron-103 | ATATTCGGTCGAAAATACCGAACGAACCACCAGGCTGGCTGA |
| | (SEQ ID NO. 103) |
| 21bpsGridiron-104 | CCTTCATCAAGTATCCAGAACAATATTACCGCCATAACCGAT |
| | (SEQ ID NO. 104) |
| 21bpsGridiron-105 | TCTTTAGGAGCGGTGTACAGACCAGGCGCATAGCAGAAGATA |
| | (SEQ ID NO. 105) |
| 21bpsGridiron-106 | AAACAGAGGTGGCTCATTCAGTGAATAAGGCTATCTAAAATA |
| | (SEQ ID NO. 106) |
| 21bpsGridiron-107 | GTAACAAAGCTAGGCGGTCAGTATTAACACCGTGCGGAATCG |
| | (SEQ ID NO. 107) |
| 21bpsGridiron-108 | TCATAAATATTTTGCCTGAGTAGAAGAACTCACCAAATCAAC |
| | (SEQ ID NO. 108) |
| 21bpsGridiron-109 | AAATCAACAGTAGACTGGATAGCGTCCAATACCCTGCAACAG |
| | (SEQ ID NO. 109) |
| 21bpsGridiron-110 | TGCCACGCTGAAATCAAAAATCAGGTCTTTACGTCAGTTGGC |
| | (SEQ ID NO. 110) |
| 21bpsGridiron-111 | GAATGACCATAGAGCCAGCAGCAAATGAAAAATGGCATCAAT |
| | (SEQ ID NO. 111) |
| 21bpsGridiron-112 | TCTACTAATAGTAACCGTTGTAGCAATACTTCCAGAAAACGA |
| | (SEQ ID NO. 112) |
| 21bpsGridiron-113 | TATATTTTCATCAAACCCTCAATCAATATCTGCCTGACTATT |
| | (SEQ ID NO. 113) |
| 21bpsGridiron-114 | ATAGTCAGAAGAATATACAGTAACAGTACCTTCCTGTTTAGC |
| | (SEQ ID NO. 114) |
| 21bpsGridiron-115 | GTAAAATGTTTTGAAAGGAATTGAGGAAGGTTTGCCCTGACG |
| | (SEQ ID NO. 115) |
| 21bpsGridiron-116 | AGAAACACCAGAAATAAAGAAATTGCGTAGATGGGGGTAATA |
| | (SEQ ID NO. 116) |
| 21bpsGridiron-117 | ATGATGAAACAAAGGGAACCGAACTGACCAACAATTATTTGC |
| | (SEQ ID NO. 117) |
| 21bpsGridiron-118 | ACGTAAAACAGAACGAGTAGTAAATTGGGCTTGCAAAAGAAG |
| | (SEQ ID NO. 118) |
| 21bpsGridiron-119 | GCAGAGGCGAATGCAAAAGAAGTTTTGCCAGATTTCAGGTTT |
| | (SEQ ID NO. 119) |
| 21bpsGridiron-120 | AACGTCAGATGCAAAGCGGATTGCATCAAAAAACAAAATCGC |
| | (SEQ ID NO. 120) |

TABLE 2
Sequences of the staples in the 42 bps Gridiron structure

| Name (No.) | Sequence |
|---|---|
| 42bpsGridiron-1 | CCTCCCGACTTGCGGGAGGTTCTGCATTAATGAATCGGCCAA (SEQ ID NO. 121) |
| 42bpsGridiron-2 | TAACTCACATTAATTGCGTTGAGAATTAACTGAACACCCTGA (SEQ ID NO. 122) |
| 42bpsGridiron-3 | AAAATGAAAATAGCAGCCTTTTTAAATTTTTGTTAAATCAGC (SEQ ID NO. 123) |
| 42bpsGridiron-4 | AACAGGAAGATTGTATAAGCATACAATTTTATCCTGAATCTT (SEQ ID NO. 124) |
| 42bpsGridiron-5 | AGTTGCTATTTTGCACCCAGCAATATTTAAATTGTAAACGTT (SEQ ID NO. 125) |
| 42bpsGridiron-6 | AATATTTTGTTAAAATTCGCAACAGAGAGAATAACATAAAAA (SEQ ID NO. 126) |
| 42bpsGridiron-7 | CAGGGAAGCGCATTAGACGGGCGCTCACTGCCCGCTTTCCAG (SEQ ID NO. 127) |
| 42bpsGridiron-8 | TCGGGAAACCTGTCGTGCCAGTTGAAGCCTTAAATCAAGATT (SEQ ID NO. 128) |
| 42bpsGridiron-9 | ACCAACGCTAACGAGCGTCTTTGTCAATCATATGTACCCCGG (SEQ ID NO. 129) |
| 42bpsGridiron-10 | GGTCATTGCCTGAGAGTCTGGACGATTTTTTGTTTAACGTCA (SEQ ID NO. 130) |
| 42bpsGridiron-11 | TTATCCCAATCCAAATAAGAAAGCAAACAAGAGAATCGATGA (SEQ ID NO. 131) |
| 42bpsGridiron-12 | ACGGTAATCGTAAAACTAGCATCCAGAGCCTAATTTGCCAGT (SEQ ID NO. 132) |
| 42bpsGridiron-13 | CCGCCACCCTCAGAGCCACCATTTCATCAACATTAAATGTGA (SEQ ID NO. 133) |
| 42bpsGridiron-14 | TCATTTTTTAACCAATAGGAAGTAGCGCGTTTTCATCGGCAT (SEQ ID NO. 134) |
| 42bpsGridiron-15 | AACCATCGATAGCAGCACCGTTGGGGTGCCTAATGAGTGAGC (SEQ ID NO. 135) |
| 42bpsGridiron-16 | AGCTTGCATGCCTGCAGGTCGTAGTTGCGCCGACAATGACAA (SEQ ID NO. 136) |
| 42bpsGridiron-17 | TTTCGGTCATAGCCCCCTTATAGAGATCTACAAAGGCTATCA (SEQ ID NO. 137) |
| 42bpsGridiron-18 | CCTCATATATTTTAAATGCAAAAAAAAGGCTCCAAAAGGAGC (SEQ ID NO. 138) |
| 42bpsGridiron-19 | TTTCACGTTGAAAATCTCCAATGCCTGAGTAATGTGTAGGTA (SEQ ID NO. 139) |
| 42bpsGridiron-20 | AAGATTCAAAAGGGTGAGAAATGAGAATAGAAAGGAACAACT (SEQ ID NO. 140) |
| 42bpsGridiron-21 | TCATAGTTAGCGTAACGATCTTGGTCATAGCTGTTTCCTGTG (SEQ ID NO. 141) |
| 42bpsGridiron-22 | CCGAGCTCGAATTCGTAATCAAAAGTTTTGTCGTCTTTCCAG (SEQ ID NO. 142) |
| 42bpsGridiron-23 | ACGTTAGTAAATGAATTTTCTTCTCCGTGGGAACAAACGGCG (SEQ ID NO. 143) |
| 42bpsGridiron-24 | GCGAGTAACAACCCGTCGGATGTATGGGATTTTGCTAAACAA (SEQ ID NO. 144) |
| 42bpsGridiron-25 | CTTTAATTGTATCGGTTTATCTCACGTTGGTGTAGATGGGCG (SEQ ID NO. 145) |
| 42bpsGridiron-26 | GATTGACCGTAATGGGATAGGAGCTTGCTTTCGAGGTGAATT (SEQ ID NO. 146) |
| 42bpsGridiron-27 | CTTTCAACAGTTTCAGCGGAGGGCCGGAGACAGTCAAATCAC (SEQ ID NO. 147) |
| 42bpsGridiron-28 | CATCAATATGATATTCAACCGTCAGAGCCGCCACCCTCAGAA (SEQ ID NO. 148) |
| 42bpsGridiron-29 | CCACCACCGGAACCGCCTCCCTTCTAGCTGATAAATTAATGC (SEQ ID NO. 149) |
| 42bpsGridiron-30 | TGAAATTGTTATCCGCTCACAGCATTGACAGGAGGTTGAGGC (SEQ ID NO. 150) |
| 42bpsGridiron-31 | CCACCACCAGAGCCGCCGCCAATTCCACACAACATACGAGCC (SEQ ID NO. 151) |
| 42bpsGridiron-32 | TCTGGCCTTCCTGTAGCCAGCCCCTCAGAGCCGCCACCAGAA (SEQ ID NO. 152) |
| 42bpsGridiron-33 | CGGAGAGGGTAGCTATTTTTGTAGCGTTTGCCATCTTTTCAT (SEQ ID NO. 153) |
| 42bpsGridiron-34 | TCTTAAACAGCTTGATACCGAACTCTAGAGGATCCCCGGGTA (SEQ ID NO. 154) |
| 42bpsGridiron-35 | GGAAGCATAAAGTGTAAAGCCAATCAGTAGCGACAGAATCAA (SEQ ID NO. 155) |
| 42bpsGridiron-36 | GTTTGCCTTTAGCGTCAGACTCGCCATCAAAAATAATTCGCG (SEQ ID NO. 156) |
| 42bpsGridiron-37 | ACAGGTAGAAAGATTCATCAGACTCCAGCCAGCTTTCCGGCA (SEQ ID NO. 157) |
| 42bpsGridiron-38 | CATCGTAACCGTGCATCTGCCTGGTTTAATTTCAACTTTAAT (SEQ ID NO. 158) |
| 42bpsGridiron-39 | ATTCAGTGAATAAGGCTTGCCGTAAAACGACGGCCAGTGCCA (SEQ ID NO. 159) |
| 42bpsGridiron-40 | CATTGTGAATTACCTTATGCGAAGGATAAAAATTTTTAGAAC (SEQ ID NO. 160) |
| 42bpsGridiron-41 | TAGCAAAATTAAGCAATAAAGTCTACTAATAGTAGTAGCATT (SEQ ID NO. 161) |
| 42bpsGridiron-42 | CGAACGAGTAGATTTAGTTTGCGCTATTACGCCAGCTGGCGA (SEQ ID NO. 162) |
| 42bpsGridiron-43 | GGCGATCGGTGCGGGCCTCTTACCATTAGATACATTTCGCAA (SEQ ID NO. 163) |
| 42bpsGridiron-44 | ATGGTCAATAACCTGTTTAGCAGGCAAAGCGCCATTCGCCAT (SEQ ID NO. 164) |
| 42bpsGridiron-45 | CCGCTTCTGGTGCCGGAAACCTATATTTTCATTTGGGGCGCG (SEQ ID NO. 165) |
| 42bpsGridiron-46 | AGCTGAAAAGGTGGCATCAATCCTCAGAGCATAAAGCTAAAT (SEQ ID NO. 166) |
| 42bpsGridiron-47 | CGGTTGTACCAAAAACATTATAACTAACGGAACAACATTATT (SEQ ID NO. 167) |
| 42bpsGridiron-48 | AAAATCTACGTTAATAAAACGGACCCTGTAATACTTTTGCGG (SEQ ID NO. 168) |
| 42bpsGridiron-49 | AAGGGGGATGTGCTGCAAGGCACGCCAAAAGGAATTACGAGG (SEQ ID NO. 169) |
| 42bpsGridiron-50 | TTCAACTAATGCAGATACATAGATTAAGTTGGGTAACGCCAG (SEQ ID NO. 170) |
| 42bpsGridiron-51 | TATCGGCCTCAGGAAGATCGCTTGAGATTTAGGAATACCACA (SEQ ID NO. 171) |
| 42bpsGridiron-52 | GAGAAGCCTTTATTTCAACGCATTTTAAGAACTGGCTCATTA (SEQ ID NO. 172) |
| 42bpsGridiron-53 | GGTTTTCCCAGTCACGACGTTCTGACGAGAAACACCAGAACG (SEQ ID NO. 173) |
| 42bpsGridiron-54 | AGTAGTAAATTGGGCTTGAGAAGTTTGAGGGGACGACGACAG (SEQ ID NO. 174) |
| 42bpsGridiron-55 | TCTTTCCTTATCATTCCAAGACGTAAAACAGAAATAAAGAAA (SEQ ID NO. 175) |
| 42bpsGridiron-56 | TTGTTTGGATTATACTTCTGAAAAGTTACCAGAAGGAAACCG (SEQ ID NO. 176) |
| 42bpsGridiron-57 | AATGAAATAGCAATAGCTATCAATGGATTATTTACATTGGCA (SEQ ID NO. 177) |
| 42bpsGridiron-58 | CCAGCCATTGCAACAGGAAAAGCCGTTTTTATTTTCATCGTA (SEQ ID NO. 178) |
| 42bpsGridiron-59 | GCACTCATCGAGAACAAGCAAACGCTCATGGAAATACCTACA (SEQ ID NO. 179) |
| 42bpsGridiron-60 | TTTTGACGCTCAATCGTCTGATTACCGAAGCCCTTTTTAAGA (SEQ ID NO. 180) |
| 42bpsGridiron-61 | AAAGTAAGCAGATAGCCGAACATAATGGAAGGGTTAGAACCT (SEQ ID NO. 181) |
| 42bpsGridiron-62 | ACCATATCAAAATTATTTGCAACGGGTATTAAACCAAGTACC (SEQ ID NO. 182) |
| 42bpsGridiron-63 | GGAATCATTACCGCGCCCAATTCAAACTATCGGCCTTGCTGG (SEQ ID NO. 183) |
| 42bpsGridiron-64 | AATTAACCGTTGTAGCAATACCCAATAATAAGAGCAAGAAAC (SEQ ID NO. 184) |
| 42bpsGridiron-65 | ACAAAGTCAGAGGGTAATTGACCGCCTGGCCCTGAGAGAGTT (SEQ ID NO. 185) |
| 42bpsGridiron-66 | TATTGGGCGCCAGGGTGGTTTAACGCGAGGCGTTTTAGCGAA (SEQ ID NO. 186) |
| 42bpsGridiron-67 | AGGCTTATCCGGTATTCTAAGTTCTTTTCACCAGTGAGACGG (SEQ ID NO. 187) |
| 42bpsGridiron-68 | GCAACAGCTGATTGCCCTTCAGCGCTAATATCAGAGAGATAA (SEQ ID NO. 188) |
| 42bpsGridiron-69 | CCCACAAGAATTGAGTTAAGCTTCTTTGATTAGTAATAACAT (SEQ ID NO. 189) |
| 42bpsGridiron-70 | CACTTGCCTGAGTAGAAGAACAGCAAGCAAATCAGATATAGA (SEQ ID NO. 190) |
| 42bpsGridiron-71 | TTCCAGTAAGCGTCATACATGTGACCTGAAAGCGTAAGAATA (SEQ ID NO. 191) |
| 42bpsGridiron-72 | GATTCACCAGTCACACGACCAAAGGTGAATTATCACCGTCAC (SEQ ID NO. 192) |
| 42bpsGridiron-73 | CAAAAGGGCGACATTCAACCGAATTCATCAATATAATCCTGA (SEQ ID NO. 193) |
| 42bpsGridiron-74 | TTTACAAACAATTCGACAACTACTTTTTCATGAGGAAGTTTC (SEQ ID NO. 194) |
| 42bpsGridiron-75 | CAACCATCGCCCACGCATAACAAAGAACGTGGACTCCAACGT (SEQ ID NO. 195) |
| 42bpsGridiron-76 | GCAGCAAGCGGTCCACGCTGGGGCCGGAAACGTCACCAATGA (SEQ ID NO. 196) |
| 42bpsGridiron-77 | CGACTTGAGCCATTTGGGAATAAAGAGTCTGTCCATCACGCA (SEQ ID NO. 197) |
| 42bpsGridiron-78 | TGGTTGCTTTGACGAGCACGTCTTTTGCGGGATCGTCACCCT (SEQ ID NO. 198) |
| 42bpsGridiron-79 | CTTGCAGGGAGTTAAAGGCCGATAACGTGCTTTCCTCGTTAG (SEQ ID NO. 199) |
| 42bpsGridiron-80 | AATCAGAGCGGGAGCTAAACACCGTAACACTGAGTTTCGTCA (SEQ ID NO. 200) |
| 42bpsGridiron-81 | GGAGGTTTAGTACCGCCACCCTGAGTAACATTATCATTTTGC (SEQ ID NO. 201) |
| 42bpsGridiron-82 | ACGTTATTAATTTTAAAAGTTTCAGAACCGCCACCCTCAGAA (SEQ ID NO. 202) |
| 42bpsGridiron-83 | CCGCCACCCTCAGAGCCACCAGAATGGCTATTAGTCTTTAAT (SEQ ID NO. 203) |
| 42bpsGridiron-84 | CGTGGCACAGACAATATTTTTCCCTCATTTTCAGGGATAGCA (SEQ ID NO. 204) |
| 42bpsGridiron-85 | CAGCAGCGAAAGACAGCATCGACATCGCCATTAAAAATACCG (SEQ ID NO. 205) |
| 42bpsGridiron-86 | GCGCGAACTGATAGCCCTAAAGAACGAGGGTAGCAACGGCTA (SEQ ID NO. 206) |
| 42bpsGridiron-87 | AGCCCAATAGGAACCCATGTAGGAGGCCGATTAAAGGGATTT (SEQ ID NO. 207) |
| 42bpsGridiron-88 | ATCAAAAGAATAGCCCGAGATGTAGCATTCCACAGACAGCCC (SEQ ID NO. 208) |
| 42bpsGridiron-89 | CCAGTACAAACTACAACGCCTAGGGTTGAGTGTTGTTCCAGT (SEQ ID NO. 209) |
| 42bpsGridiron-90 | TAGACAGGAACGGTACGCCAGGCGCAGTCTCTGAATTTACCG (SEQ ID NO. 210) |
| 42bpsGridiron-91 | AGGTCAGACGATTGGCCTTGAAATCGGCAAAATCCCTTATAA (SEQ ID NO. 211) |
| 42bpsGridiron-92 | CCTGTTTGATGGTGGTTCCGATATTCACAAACAAATAAATCC (SEQ ID NO. 212) |
| 42bpsGridiron-93 | TCATTAAAGCCAGAATGGAAAAATCCTGAGAAGTGTTTTTAT (SEQ ID NO. 213) |
| 42bpsGridiron-94 | GGAACAAAGAAACCACCAGAAGGGTCAGTGCCTTGAGTAACA (SEQ ID NO. 214) |
| 42bpsGridiron-95 | TACTGGTAATAAGTTTTAACGGGAGCGGAATTATCATCATAT (SEQ ID NO. 215) |
| 42bpsGridiron-96 | CCAACAGAGATAGAACCCTTCGCTTTTGATGATACAGGAGTG (SEQ ID NO. 216) |
| 42bpsGridiron-97 | AATCAGTGAGGCCACCGAGTATAGAGCCAGCAAAATCACCAG (SEQ ID NO. 217) |
| 42bpsGridiron-98 | TAGCACCATTACCATTAGCAATTTGCCCCAGCAGGCGAAAAT (SEQ ID NO. 218) |
| 42bpsGridiron-99 | TTGGAACAAGAGTCCACTATTCGATATATTCGGTCGCTGAGG (SEQ ID NO. 219) |
| 42bpsGridiron-100 | CAGAGGCTTTGAGGACTAAAGCGTATTAAATCCTTTGCCCGA (SEQ ID NO. 220) |
| 42bpsGridiron-101 | TCCTGATTATCAGATGATGGCATTGAGGGAGGGAAGGTAAAT (SEQ ID NO. 221) |
| 42bpsGridiron-102 | ATTGACGGAAATTATTCATTAGTAATAAAAGGGACATTCTGG (SEQ ID NO. 222) |
| 42bpsGridiron-103 | TTTGCCAGAGGGGGTAATAGTGTGCCACGCTGAGAGCCAGCA (SEQ ID NO. 223) |
| 42bpsGridiron-104 | AACGAACCACCAGCAGAAGATATGAACGGTGTACAGACCAGG (SEQ ID NO. 224) |
| 42bpsGridiron-105 | CGGAACGAGGCGCAGACGGTCGAGGATTTAGAAGTATTAGAC (SEQ ID NO. 225) |
| 42bpsGridiron-106 | CAAAGGGCGAAAAACCGTCTAATCAACGTAACAAAGCTGCTC (SEQ ID NO. 226) |
| 42bpsGridiron-107 | CGCATAGGCTGGCTGACCTTCGCCGCTACAGGGCGCGTACTA (SEQ ID NO. 227) |
| 42bpsGridiron-108 | CGTGGCGAGAAAGGAAGGGAAATATGCAACTAAAGTACGGTG (SEQ ID NO. 228) |
| 42bpsGridiron-109 | AGGATTAGAGAGTACCTTTAAGAAAGGAATTGAGGAAGGTTA (SEQ ID NO. 229) |
| 42bpsGridiron-110 | TCAGTTGGCAAATCAACAGTTTTGCTCCTTTTGATAAGAGGT (SEQ ID NO. 230) |
| 42bpsGridiron-111 | CATTTTTGCGGATGGCTTAGATCACCTTGCTGAACCTCAAAT (SEQ ID NO. 231) |
| 42bpsGridiron-112 | GCAAATGAAAAATCTAAAGCAGCTTAATTGCTGAATATAATG (SEQ ID NO. 232) |
| 42bpsGridiron-113 | CTGTAGCTCAACATGTTTTAAGAAAGCGAAAGGAGCGGGCGC (SEQ ID NO. 233) |
| 42bpsGridiron-114 | TAAAGCACTAAATCGGAACCCAACAGTTGATTCCCAATTCTG (SEQ ID NO. 234) |
| 42bpsGridiron-115 | TCTGGAAGTTTCATTCCATATTAAAGGGAGCCCCCGATTTAG (SEQ ID NO. 235) |
| 42bpsGridiron-116 | TAGGGCGCTGGCAAGTGTAGCAGAGGCTTTTGCAAAAGAAGT (SEQ ID NO. 236) |
| 42bpsGridiron-117 | CATAGTAAGAGCAACACTATCTTTTTTGGGGTCGAGGTGCCG (SEQ ID NO. 237) |
| 42bpsGridiron-118 | TGAACCATCACCCAAATCAAGATAACCCTCGTTTACCAGACG (SEQ ID NO. 238) |
| 42bpsGridiron-119 | ACGATAAAAACCAAAATAGCGGGTCACGCTGCGCGTAACCAC (SEQ ID NO. 239) |
| 42bpsGridiron-120 | TCTAAAATATCTTTAGGAGCAATAAATATTCATTGAATCCCC (SEQ ID NO. 240) |
| 42bpsGridiron-121 | GTCCAATACTGCGGAATCGTCCTAACAACTAATAGATTAGAG (SEQ ID NO. 241) |
| 42bpsGridiron-122 | GTATTAACACCGCCTGCAACAAAAATGTTTAGACTGGATAGC (SEQ ID NO. 242) |
| 42bpsGridiron-123 | CACACCCGCCGCGCTTAATGCATCAAGAGTAATCTTGACAAG (SEQ ID NO. 243) |
| 42bpsGridiron-124 | AACCGGATATTCATTACCCAATCAGGGCGATGGCCCACTACG (SEQ ID NO. 244) |
| 42bpsGridiron-125 | CCGTCAATAGATAATACATTTAATCATAAGGGAACCGAACTG (SEQ ID NO. 245) |
| 42bpsGridiron-126 | ACCAACTTTGAAAGAGGACAGAAAACAGAGGTGAGGOGGTCA (SEQ ID NO. 246) |
| 42bpsGridiron-127 | ATCAACAATAGATAAGTCCTGTGTCCAGACGACGACAATAAA (SEQ ID NO. 247) |
| 42bpsGridiron-128 | GCAGAGGCATTTTCGAGCCAGGTATGTTAGCAAACGTAGAAA (SEQ ID NO. 248) |
| 42bpsGridiron-129 | AGGAAACGCAATAATAACGGATTGCTTTGAATACCAAGTTAC (SEQ ID NO. 249) |
| 42bpsGridiron-130 | GTCAGATGAATATACAGTAACAAACCAATCAATAATCGGCTG (SEQ ID NO. 250) |
| 42bpsGridiron-131 | TCCTAATTTACGAGCATGTAGAGTACCTTTTACATCGGGAGA (SEQ ID NO. 251) |
| 42bpsGridiron-132 | AACAATAACGGATTCGCCTGAATACCCAAAAGAACTGGCATG (SEQ ID NO. 252) |
| 42bpsGridiron-133 | ATTAAGACTCCTTATTACGCATAATAAGAGAATATAAAGTAC (SEQ ID NO. 253) |
| 42bpsGridiron-134 | CGACAAAAGGTAAAGTAATTCAACAAGAAAAATAATATCCCA (SEQ ID NO. 254) |
| 42bpsGridiron-135 | CATTAAACGGGTAAAATACGTTGAGTGAATAACCTTGCTTCT (SEQ ID NO. 255) |
| 42bpsGridiron-136 | AAAATCGCGCAGAGGCGAATTATGGTTTACCAGCGCCAAAGA (SEQ ID NO. 256) |
| 42bpsGridiron-137 | ATAAAAGAAACGCAAAGACACCAACGCCAACATGTAATTTAG (SEQ ID NO. 257) |
| 42bpsGridiron-138 | GTGATAAATAAGGCGTTAAATAGAATACACTAAAACACTCAT (SEQ ID NO. 258) |
| 42bpsGridiron-139 | ACCTAAAACGAAAGAGGCAAAAAGAATAAACACCGGAATCAT (SEQ ID NO. 259) |
| 42bpsGridiron-140 | AATTACTAGAAAAAGCCTGTTGGATAAGTGCCGTCGAGAGGG (SEQ ID NO. 260) |
| 42bpsGridiron-141 | GGGTTTTGCTCAGTACCAGGCTAGTATCATATGCGTTATACA (SEQ ID NO. 261) |
| 42bpsGridiron-142 | TACATTTAACAATTTCATTTGATAGGTGTATCACCGTACTCA (SEQ ID NO. 262) |
| 42bpsGridiron-143 | TTGATATAAGTATAGCCCGGAAATTACCTTTTTTAATGGAAA (SEQ ID NO. 263) |
| 42bpsGridiron-144 | AATTCTTACCAGTATAAAGCCGTATTAAGAGGCTGAGACTCC (SEQ ID NO. 264) |
| 42bpsGridiron-145 | GTGCCCGTATAAACAGTTAATCATCAAGAAAACAAAATTAAT (SEQ ID NO. 265) |
| 42bpsGridiron-146 | AAAAGAAGATGATGAAACAAAGCCCCCTGCCTATTTCGGAAC (SEQ ID NO. 266) |
| 42bpsGridiron-147 | CTATTATTCTGAAACATGAAAAACGCTCAACAGTAGGGCTTA (SEQ ID NO. 267) |
| 42bpsGridiron-148 | ATTGAGAATCGCCATATTTAACACGGAATAAGTTTATTTTGT (SEQ ID NO. 268) |
| 42bpsGridiron-149 | CACAATCAATAGAAAATTCATATTCATTTCAATTACCTGAGC (SEQ ID NO. 269) |
| 42bpsGridiron-150 | CAGTACATAAATCAATATATGAATGCCACTACGAAGGCACCA (SEQ ID NO. 270) |
| 42bpsGridiron-151 | GTAAATCGTCGCTATTAATTAACCTGCTCCATGTTACTTAGC (SEQ ID NO. 271) |
| 42bpsGridiron-152 | AGCGCGAAACAAAGTACAACGATGGTTTGAAATACCGACCGT (SEQ ID NO. 272) |
| 42bpsGridiron-153 | TATAACTATATGTAAATGCTGCAAATATCGCGTTTTAATTCG (SEQ ID NO. 273) |
| 42bpsGridiron-154 | AAGAGGAAGCCCGAAAGACTTATGCAAATCCAATCGCAAGAC (SEQ ID NO. 274) |
| 42bpsGridiron-155 | TAGTTGAATTTATCAAAATCATGGAAGCAAACTCCAACAGGTC (SEQ ID NO. 275) |
| 42bpsGridiron-156 | AGCTTCAAAGCGAACCAGACCAGGTCTGAGAGACTACCTTTT (SEQ ID NO. 276) |
| 42bpsGridiron-157 | AAAGAACGCGAGAAAACTTTTCTGACTATTATAGTCAGAAGC (SEQ ID NO. 277) |
| 42bpsGridiron-158 | CTCAAATGCTTTAAACAGTTCTAAGACGCTGAGAAGAGTCAA (SEQ ID NO. 278) |
| 42bpsGridiron-159 | AAACATAGCGATAGCTTAGATAGAAAACGAGAATGACCATAA (SEQ ID NO. 279) |
| 42bpsGridiron-160 | ATCAAAAATCAGGTCTTTACCTCAAATATATTTTAGTTAATT (SEQ ID NO. 280) |
| 42bpsGridiron-161 | TCATCTTCTGACCTAAATTTAGAGATTTGTATCATCGCCTGA (SEQ ID NO. 281) |
| 42bpsGridiron-162 | TAAATTGTGTCGAAATCCGCGATTTTCCCTTAGAATCCTTGA (SEQ ID NO. 282) |

The claims are not intended to be limited to the embodiments and examples described herein.

We claim:

1.-19. (canceled)

20. A method of forming a DNA gridiron unit, comprising:
combining a plurality of scaffold strands and staple strands complementary to said scaffold strand under conditions suitable for the formation of four four-arm junctions linked together in a two-layer square frame in which the helices on opposite sides lie in the same plane.

21. The method of claim 20, wherein said scaffold strands comprise single stranded M13mp18 DNA.

22. The method of claim 20, wherein said scaffold strands are combined with a molar excess of staple strands in TAE Mg2+ buffer.

23. The method of claim 20, wherein said scaffold strands and staple strands are annealed from 95° C. to 4° C. for a selected time.

24. The method of claim 23, wherein the selected time is: 95 to 86° C. at 4° C. per 5 minutes; 85 to 70° C. at 1° C. per 5 minutes; 70 to 40° C. at 1° C. per 15 minutes; then 40 to 25° C. at 1° C. per 10 minutes.

25. The method of claim 23, wherein the selected time is: 95 to 76° C. at 2° C. per 5 minutes; then 76 to 24° C. at 4° C. per 5 minutes.

26. A method of forming a DNA origami nanostructure, comprising:
linking a plurality of DNA gridiron units of a selected sequence of nucleotides under conditions suitable to form a 2D or 3D lattice of a selected shape.

27. The method of claim 26, wherein the selected shape is a S-shape, sphere, or screw.

28. A DNA nanostructure comprising
a plurality of immobile Holliday junction analogs linked together in a plurality of layered frames, wherein each layer of frame has at least two DNA helices which lie on opposite sides of the Holliday junction which also lie in the same plane, and wherein said plurality of immobile Holliday junction analogs are linked together with a central strand of single-stranded DNA within said layer of frame,
wherein each of the plurality of immobile Holliday junction analogs is independently separated by a selected distance, and
wherein one or more of the plurality of immobile Holliday junction analogs together form a curvilinear surface.

29. The DNA nanostructure of claim 28, wherein a layer of frame is distorted along the plane of the layer.

30. The DNA nanostructure of claim 28, wherein the distance between the Holliday junctions is progressively adjusted between junctions in latitudinal directions.

31. The DNA nanostructure of claim 28, wherein the DNA nanostructure is in the shape of a sphere, S-shaped structure, or screw.

32. The DNA nanostructure of claim 31, wherein the DNA nanostructure is in the shape of a sphere.

33. The DNA nanostructure of claim 32, wherein sphere defines an interstitial space confined by a cavity.

34. The DNA nanostructure of claim 28, wherein said single stranded DNA comprises M13mp18 DNA.

35. The DNA nanostructure of claim 34, further comprising one or a plurality of staple strand sequences.

36. The DNA nanostructure of claim 35, wherein the staple strand sequences are independently from 40 to 45 nucleotides in length.


SEQUENCE LISTING (282 sequences)

SEQ ID NO: 1 - DNA, 42 bp, synthetic construct
  misc_feature 1..42: note = Staple Strand
  source 1..42: mol_type = other DNA
gaaaattcataagtaagcgtcatacatggcttagacgggaga

SEQ ID NO: 2 - DNA, 42 bp, synthetic construct
  misc_feature 1..42: note = Staple Strand
  source 1..42: mol_type = other DNA
atattcacaaaataaaaacagggaagcgcattttgatgatac

SEQ ID NO: 3 - DNA, 42 bp, synthetic construct
  misc_feature 1..42: note = Staple Strand
  source 1..42: mol_type = other DNA
aggagtgtactaataaacagccatattatttaattggccttg

SEQ ID NO: 4 - DNA, 42 bp, synthetic construct
  misc_feature 1..42: note = Staple Strand
  source 1..42: mol_type = other DNA
gccagttacaaggtaataagttttaacggggtgtcctgaaca

SEQ ID NO: 5 - DNA, 42 bp, synthetic construct
  misc_feature 1..42: note = Staple Strand
  source 1..42: mol_type = other DNA
gcagaacgcgcgaggttgaggcaggtcagacgtcccaatcca

SEQ ID NO: 6 - DNA, 42 bp, synthetic construct
  misc_feature 1..42: note = Staple Strand
  source 1..42: mol_type = other DNA
aataagaaacggacttgagccatttgggaattttcagctaat

SEQ ID NO: 7 - DNA, 42 bp, synthetic construct
  misc_feature 1..42: note = Staple Strand
  source 1..42: mol_type = other DNA
gagagaataaccaaataaatcctcattaaagcaaaagggcga

SEQ ID NO: 8 - DNA, 42 bp, synthetic construct
  misc_feature 1..42: note = Staple Strand
  source 1..42: mol_type = other DNA
agcattgacagctgtttatcaacaatagataacagtgccttg

SEQ ID NO: 9 - DNA, 42 bp, synthetic construct
  misc_feature 1..42: note = Staple Strand
  source 1..42: mol_type = other DNA
agtaacagtgcccagtaataagagaatataaaagccgccgcc

SEQ ID NO: 10 - DNA, 42 bp, synthetic construct
  misc_feature 1..42: note = Staple Strand
  source 1..42: mol_type = other DNA
gcattttcgagccgtataaacagttaatgccctatcaaaatc

SEQ ID NO: 11 - DNA, 42 bp, synthetic construct
  misc_feature 1..42: note = Staple Strand
  source 1..42: mol_type = other DNA
attaagacgctcgccaccagaaccaccaccaggtaccgacaa

SEQ ID NO: 12 - DNA, 42 bp, synthetic construct
  misc_feature 1..42: note = Staple Strand
  source 1..42: mol_type = other DNA
aaggtaaagtaagcaccattaccattagcaaggatagcttag

SEQ ID NO: 13 - DNA, 44 bp, synthetic construct
  misc_feature 1..44: note = Staple Strand
  source 1..44: mol_type = other DNA
tttaccgttcctggtttaccagcgccaaagaccagaatggaaag

SEQ ID NO: 14 - DNA, 42 bp, synthetic construct
  misc_feature 1..42: note = Staple Strand
  source 1..42: mol_type = other DNA
cattcaaccgattgacggaaattattcattaaagcctttaca

SEQ ID NO: 15 - DNA, 40 bp, synthetic construct
  misc_feature 1..40: note = Staple Strand
  source 1..40: mol_type = other DNA
gaaaatagcaggtgaattatcaccgtcaccattttttgtt

SEQ ID NO: 16 - DNA, 42 bp, synthetic construct
  misc_feature 1..42: note = Staple Strand
  source 1..42: mol_type = other DNA
gcaaagacaccgtaaatgaattttctgtatggtaattgagcg

SEQ ID NO: 17 - DNA, 42 bp, synthetic construct
  misc_feature 1..42: note = Staple Strand
  source 1..42: mol_type = other DNA
tagcattccacaccctgaacaaagtcagaggggattttgcta

SEQ ID NO: 18 - DNA, 42 bp, synthetic construct
  misc_feature 1..42: note = Staple Strand
  source 1..42: mol_type = other DNA
aacaactttcacgctaacgagcgtctttccagacaacgcctg

SEQ ID NO: 19 - DNA, 42 bp, synthetic construct
  misc_feature 1..42: note = Staple Strand
  source 1..42: mol_type = other DNA
aatcttaccaaacagtttcagcggagtgagaaatgtagaaac

SEQ ID NO: 20 - DNA, 42 bp, synthetic construct
  misc_feature 1..42: note = Staple Strand
  source 1..42: mol_type = other DNA
agaaaaataatgtttcgtcaccagtacaaactagcctaattt

SEQ ID NO: 21 - DNA, 42 bp, synthetic construct
  misc_feature 1..42: note = Staple Strand
  source 1..42: mol_type = other DNA
attaactgaacagacagccctcatagttagcgacaatcaata

SEQ ID NO: 22 - DNA, 40 bp, synthetic construct
  misc_feature 1..40: note = Staple Strand
  source 1..40: mol_type = other DNA
aacaacatgagagccagcaaaatcaccagtattctgtcca

SEQ ID NO: 23 - DNA, 42 bp, synthetic construct
  misc_feature 1..42: note = Staple Strand
  source 1..42: mol_type = other DNA
cgtaacactgaatcccatcctaatttacgagctagaaaggaa

SEQ ID NO: 24 - DNA, 42 bp, synthetic construct
  misc_feature 1..42: note = Staple Strand
  source 1..42: mol_type = other DNA
caactaaaggattaacaacgccaacatgtaatacccatgtac

SEQ ID NO: 25 - DNA, 42 bp, synthetic construct
  misc_feature 1..42: note = Staple Strand
  source 1..42: mol_type = other DNA
aatcgccatatattgcgaataataattttttcgcttaggttg

SEQ ID NO: 26 - DNA, 42 bp, synthetic construct
  misc_feature 1..42: note = Staple Strand
  source 1..42: mol_type = other DNA
ataggtctgaggggatagcaagcccaataggattaggcagag

SEQ ID NO: 27 - DNA, 44 bp, synthetic construct
  misc_feature 1..44: note = Staple Strand
  source 1..44: mol_type = other DNA
tccagacgttaacggaataagtttattttgtctaacgatctaaa

SEQ ID NO: 28 - DNA, 40 bp, synthetic construct
  misc_feature 1..40: note = Staple Strand
  source 1..40: mol_type = other DNA
tcgcccacgcagccattgcaacaggaaaaatgcgccgaca

SEQ ID NO: 29 - DNA, 42 bp, synthetic construct
  misc_feature 1..42: note = Staple Strand
  source 1..42: mol_type = other DNA
cctcattttcaagactacctttttaacctccgacgttgaaaa

SEQ ID NO: 30 - DNA, 42 bp, synthetic construct
  misc_feature 1..42: note = Staple Strand
  source 1..42: mol_type = other DNA
tctccaaaaaatgaattaccttttttaatggagagccaccac

SEQ ID NO: 31 - DNA, 40 bp, synthetic construct
  misc_feature 1..40: note = Staple Strand
  source 1..40: mol_type = other DNA
atataagtatttgacgctcaatcgtctgaagataagtgcc

SEQ ID NO: 32 - DNA, 42 bp, synthetic construct
  misc_feature 1..42: note = Staple Strand
  source 1..42: mol_type = other DNA
accctcagagcgagaagagtcaatagtgaattcctgcctatt

SEQ ID NO: 33 - DNA, 42 bp, synthetic construct
  misc_feature 1..42: note = Staple Strand
  source 1..42: mol_type = other DNA
tcggaacctattgtgagtgaataaccttgcttcagagccacc

SEQ ID NO: 34 - DNA, 40 bp, synthetic construct
  misc_feature 1..40: note = Staple Strand
  source 1..40: mol_type = other DNA
cgtttgccaattcaccagtcacacgaccagttcggtcata

SEQ ID NO: 35 - DNA, 40 bp, synthetic construct
  misc_feature 1..40: note = Staple Strand
  source 1..40: mol_type = other DNA
aaacatagcgccggaaacgtcaccaatgaataattttccc

SEQ ID NO: 36 - DNA, 42 bp, synthetic construct
  misc_feature 1..42: note = Staple Strand
  source 1..42: mol_type = other DNA
acaatttcattaaggctccaaaaggagccttttatacttctg

SEQ ID NO: 37 - DNA, 40 bp, synthetic construct
  misc_feature 1..40: note = Staple Strand
  source 1..40: mol_type = other DNA
gaacaaagaatcagtagcgacagaatcaagttgagtaaca

SEQ ID NO: 38 - DNA, 42 bp, synthetic construct
  misc_feature 1..42: note = Staple Strand
  source 1..42: mol_type = other DNA
gataatacatttgctttcgaggtgaatttcttagccctaaaa

SEQ ID NO: 39 - DNA, 40 bp, synthetic construct
  misc_feature 1..40: note = Staple Strand
  source 1..40: mol_type = other DNA
acagagatatagcgcgttttcatcggcatttaataaaagg

SEQ ID NO: 40 - DNA, 42 bp, synthetic construct
  misc_feature 1..42: note = Staple Strand
  source 1..42: mol_type = other DNA
attttaaaagttttgcctttagcgtcagactggaacccttct

SEQ ID NO: 41 - DNA, 42 bp, synthetic construct
  misc_feature 1..42: note = Staple Strand
  source 1..42: mol_type = other DNA
gacctgaaagccggaaccagagccaccaccggaacgttatta

SEQ ID NO: 42 - DNA, 42 bp, synthetic construct
  misc_feature 1..42: note = Staple Strand
  source 1..42: mol_type = other DNA
atcaaaatcacgtaagaatacgtggcacagacggttttgctc

SEQ ID NO: 43 - DNA, 42 bp, synthetic construct
  misc_feature 1..42: note = Staple Strand
  source 1..42: mol_type = other DNA
agtaccaggcgatggattatttacattggcagtcttttcata

SEQ ID NO: 44 - DNA, 42 bp, synthetic construct
  misc_feature 1..42: note = Staple Strand
  source 1..42: mol_type = other DNA
aattcgacaacgagaaggattaggattagcggaatatttttg

SEQ ID NO: 45 - DNA, 42 bp, synthetic construct
  misc_feature 1..42: note = Staple Strand
  source 1..42: mol_type = other DNA
aatggctattaccgtactcaggaggtttagtactttacaaac

SEQ ID NO: 46 - DNA, 42 bp, synthetic construct
  misc_feature 1..42: note = Staple Strand
  source 1..42: mol_type = other DNA
taggtgtatcagtctttaatgcgcgaactgataaacagcttg

SEQ ID NO: 47 - DNA, 42 bp, synthetic construct
  misc_feature 1..42: note = Staple Strand
  source 1..42: mol_type = other DNA
ataccgatagtcgctcatggaaatacctacattagcccggaa

SEQ ID NO: 48 - DNA, 42 bp, synthetic construct
  misc_feature 1..42: note = Staple Strand
  source 1..42: mol_type = other DNA
gtttatcagcttgaggatttagaagtattagaccgccaccct

SEQ ID NO: 49 - DNA, 42 bp, synthetic construct
  misc_feature 1..42: note = Staple Strand
  source 1..42: mol_type = other DNA
cagaaccgccatataatcctgattgtttggataattgtatcg

SEQ ID NO: 50 - DNA, 42 bp, synthetic construct
  misc_feature 1..42: note = Staple Strand
  source 1..42: mol_type = other DNA
agactcctcaatcgtattaaatcctttgcccgaaccgcctcc

SEQ ID NO: 51 - DNA, 42 bp, synthetic construct
  misc_feature 1..42: note = Staple Strand
  source 1..42: mol_type = other DNA
ctcagagccgctatcatcatattcctgattattaagaggctg

SEQ ID NO: 52 - DNA, 42 bp, synthetic construct
  misc_feature 1..42: note = Staple Strand
  source 1..42: mol_type = other DNA
tcgctattaataccatcgatagcagcaccgtaaaccaccaga

SEQ ID NO: 53 - DNA, 42 bp, synthetic construct
  misc_feature 1..42: note = Staple Strand
  source 1..42: mol_type = other DNA
aggagcggaatcaccctcagaaccgccaccctctgtaaatcg

SEQ ID NO: 54 - DNA, 42 bp, synthetic construct
  misc_feature 1..42: note = Staple Strand
  source 1..42: mol_type = other DNA
aaatcaatatatattctgaaacatgaaagtatcagatgatgg

SEQ ID NO: 55 - DNA, 42 bp, synthetic construct
  misc_feature 1..42: note = Staple Strand
  source 1..42: mol_type = other DNA
caattcatcaaccctcagaaccgccaccctcaaacagtacat

SEQ ID NO: 56 - DNA, 42 bp, synthetic construct
  misc_feature 1..42: note = Staple Strand
  source 1..42: mol_type = other DNA
ggttatataaccggctacagaggctttgaggacttaattgag

SEQ ID NO: 57 - DNA, 40 bp, synthetic construct
  misc_feature 1..40: note = Staple Strand
  source 1..40: mol_type = other DNA
taaggcgttcccaattctgcgaacgagtagtgaaataccg

SEQ ID NO: 58 - DNA, 44 bp, synthetic construct
  misc_feature 1..44: note = Staple Strand
  source 1..44: mol_type = other DNA
gcttaattgctaacgcaataataacggaatacaggtcatttttg

SEQ ID NO: 59 - DNA, 42 bp, synthetic construct
  misc_feature 1..42: note = Staple Strand
  source 1..42: mol_type = other DNA
taatttcatctacttcaaatatcgcgttttaatcataattac

SEQ ID NO: 60 - DNA, 42 bp, synthetic construct
  misc_feature 1..42: note = Staple Strand
  source 1..42: mol_type = other DNA
tagaaaaagccgtttaccagacgacgataaaaatattttagt

SEQ ID NO: 61 - DNA, 44 bp, synthetic construct
  misc_feature 1..44: note = Staple Strand
  source 1..44: mol_type = other DNA
agttgagatttagactccttattacgcagtatattattacaggt

SEQ ID NO: 62 - DNA, 42 bp, synthetic construct
  misc_feature 1..42: note = Staple Strand
  source 1..42: mol_type = other DNA
aagacaaagaataatcattgtgaattaccttatacaaattct

SEQ ID NO: 63 - DNA, 42 bp, synthetic construct
  misc_feature 1..42: note = Staple Strand
  source 1..42: mol_type = other DNA
taccagtataatccatgttacttagccggaacatccaatcgc

SEQ ID NO: 64 - DNA, 44 bp, synthetic construct
  misc_feature 1..44: note = Staple Strand
  source 1..44: mol_type = other DNA
atctttgacccatacataaaggtggcaacataggcaaaagaata

SEQ ID NO: 65 - DNA, 40 bp, synthetic construct
  misc_feature 1..40: note = Staple Strand
  source 1..40: mol_type = other DNA
aaccgaggagaatataatgctgtagctcaagccgaacaaa

SEQ ID NO: 66 - DNA, 42 bp, synthetic construct
  misc_feature 1..42: note = Staple Strand
  source 1..42: mol_type = other DNA
ctaatatcagagcaccaacctaaaacgaaagataaaagaaac

SEQ ID NO: 67 - DNA, 42 bp, synthetic construct
  misc_feature 1..42: note = Staple Strand
  source 1..42: mol_type = other DNA
ccactacgaaggagataacccacaagaattgaaaacaaagta

SEQ ID NO: 68 - DNA, 42 bp, synthetic construct
  misc_feature 1..42: note = Staple Strand
  source 1..42: mol_type = other DNA
caacggagattctattttgcacccagctacaaatacgtaatg

SEQ ID NO: 69 - DNA, 40 bp, synthetic construct
  misc_feature 1..40: note = Staple Strand
  source 1..40: mol_type = other DNA
tagaaggctaagtacggtgtctggaagtttcccaatagca

SEQ ID NO: 70 - DNA, 42 bp, synthetic construct
  misc_feature 1..42: note = Staple Strand
  source 1..42: mol_type = other DNA
caatcaataatagtttccattaaacgggtaaattttatcctg

SEQ ID NO: 71 - DNA, 42 bp, synthetic construct
  misc_feature 1..42: note = Staple Strand
  source 1..42: mol_type = other DNA
tttcatgaggacggctgtctttccttatcattgtgtcgaaat

SEQ ID NO: 72 - DNA, 42 bp, synthetic construct
  misc_feature 1..42: note = Staple Strand
  source 1..42: mol_type = other DNA
ccgcgacctgcagccaacgctcaacagtagggctaaagactt

SEQ ID NO: 73 - DNA, 42 bp, synthetic construct
  misc_feature 1..42: note = Staple Strand
  source 1..42: mol_type = other DNA
aagattagttgtgtatcatcgcctgataaattccaagaacgg

SEQ ID NO: 74 - DNA, 42 bp, synthetic construct
  misc_feature 1..42: note = Staple Strand
  source 1..42: mol_type = other DNA
gtattaaaccaattataccagtcaggacgttggccttaaatc

SEQ ID NO: 75 - DNA, 42 bp, synthetic construct
  misc_feature 1..42: note = Staple Strand
  source 1..42: mol_type = other DNA
agaactggctcagtaccgcactcatcgagaacagcaacacta

SEQ ID NO: 76 - DNA, 42 bp, synthetic construct
  misc_feature 1..42: note = Staple Strand
  source 1..42: mol_type = other DNA
tcataaccctctgtttagtatcatatgcgttatgcgatttta

SEQ ID NO: 77 - DNA, 42 bp, synthetic construct
  misc_feature 1..42: note = Staple Strand
  source 1..42: mol_type = other DNA
agcgaacctccggaattacgaggcatagtaagaagcaagccg

SEQ ID NO: 78 - DNA, 42 bp, synthetic construct
  misc_feature 1..42: note = Staple Strand
  source 1..42: mol_type = other DNA
tttttattttcgaccggaagcaaactccaacagaggcgtttt

SEQ ID NO: 79 - DNA, 42 bp, synthetic construct
  misc_feature 1..42: note = Staple Strand
  source 1..42: mol_type = other DNA
aaagcgaaccaatcgtaggaatcattaccgcgcattccatat

SEQ ID NO: 80 - DNA, 42 bp, synthetic construct
  misc_feature 1..42: note = Staple Strand
  source 1..42: mol_type = other DNA
aacagttgattaaataagaataaacaccggaattcgagcttc

SEQ ID NO: 81 - DNA, 42 bp, synthetic construct
  misc_feature 1..42: note = Staple Strand
  source 1..42: mol_type = other DNA
atatgcaactatatccggtattctaagaacgcggtcaggatt

SEQ ID NO: 82 - DNA, 42 bp, synthetic construct
  misc_feature 1..42: note = Staple Strand
  source 1..42: mol_type = other DNA
agagagtaccttttaagaaaagtaagcagatacatgttttaa

SEQ ID NO: 83 - DNA, 42 bp, synthetic construct
  misc_feature 1..42: note = Staple Strand
  source 1..42: mol_type = other DNA
taacgccaaaacgacttgcgggaggttttgaaggaagaaaaa

SEQ ID NO: 84 - DNA, 42 bp, synthetic construct
  misc_feature 1..42: note = Staple Strand
  source 1..42: mol_type = other DNA
tctacgttaataagaaacaatgaaatagcaattgcagataca

SEQ ID NO: 85 - DNA, 42 bp, synthetic construct
  misc_feature 1..42: note = Staple Strand
  source 1..42: mol_type = other DNA
gtagaaaatacccagcgattataccaagcgcggttaagccca

SEQ ID NO: 86 - DNA, 42 bp, synthetic construct
  misc_feature 1..42: note = Staple Strand
  source 1..42: mol_type = other DNA
ataataagagcaaaacgaactaacggaacaacgttagcaaac

SEQ ID NO: 87 - DNA, 42 bp, synthetic construct
  misc_feature 1..42: note = Staple Strand
  source 1..42: mol_type = other DNA
tggcatgattaaggaataccacattcaactaaagctatctta

SEQ ID NO: 88 - DNA, 42 bp, synthetic construct
  misc_feature 1..42: note = Staple Strand
  source 1..42: mol_type = other DNA
ccgaagcccttttaattgctccttttgataagccaaaagaac

SEQ ID NO: 89 - DNA, 42 bp, synthetic construct
  misc_feature 1..42: note = Staple Strand
  source 1..42: mol_type = other DNA
aagcccgaaagtctgacctaaatttaatggttatttagtttg

SEQ ID NO: 90 - DNA, 42 bp, synthetic construct
  misc_feature 1..42: note = Staple Strand
  source 1..42: mol_type = other DNA
accattagatagattgctttgaataccaagttgattaagagg

SEQ ID NO: 91 - DNA, 40 bp, synthetic construct
  misc_feature 1..40: note = Staple Strand
  source 1..40: mol_type = other DNA
taaacagtttttgattagtaataacatcaccattgaatcc

SEQ ID NO: 92 - DNA, 42 bp, synthetic construct
  misc_feature 1..42: note = Staple Strand
  source 1..42: mol_type = other DNA
aatttcaacttcgcgagaaaactttttcaaataccaaaatag

SEQ ID NO: 93 - DNA, 42 bp, synthetic construct
  misc_feature 1..42: note = Staple Strand
  source 1..42: mol_type = other DNA
cgagaggctttttattcatttcaattacctgagagatggttt

SEQ ID NO: 94 - DNA, 40 bp, synthetic construct
  misc_feature 1..40: note = Staple Strand
  source 1..40: mol_type = other DNA
attcattacaactatcggccttgctggtaaagtaatcttg

SEQ ID NO: 95 - DNA, 42 bp, synthetic construct
  misc_feature 1..42: note = Staple Strand
  source 1..42: mol_type = other DNA
gagggtagcaatatatgtaaatgctgatgcaagaggcgcaga

SEQ ID NO: 96 - DNA, 42 bp, synthetic construct
  misc_feature 1..42: note = Staple Strand
  source 1..42: mol_type = other DNA
cggtcaatcataacatcaagaaaacaaaattagcatcggaac

SEQ ID NO: 97 - DNA, 40 bp, synthetic construct
  misc_feature 1..40: note = Staple Strand
  source 1..40: mol_type = other DNA
gattcgcctcatttcgcaaatggtcaataattacatcggg

SEQ ID NO: 98 - DNA, 42 bp, synthetic construct
  misc_feature 1..42: note = Staple Strand
  source 1..42: mol_type = other DNA
aataatggaagcaccctcagcagcgaaagacaattacattta

SEQ ID NO: 99 - DNA, 42 bp, synthetic construct
  misc_feature 1..42: note = Staple Strand
  source 1..42: mol_type = other DNA
tgcgggatcgtggttagaacctaccatatcaatttgaaagag

SEQ ID NO: 100 - DNA, 42 bp, synthetic construct
  misc_feature 1..42: note = Staple Strand
  source 1..42: mol_type = other DNA
gacagatgaacactaacaactaatagattagaaggccgcttt

SEQ ID NO: 101 - DNA, 40 bp, synthetic construct
  misc_feature 1..40: note = Staple Strand
  source 1..40: mol_type = other DNA
ctcaaatatttggggcgcgagctgaaaaggtctaaagcat

SEQ ID NO: 102 - DNA, 42 bp, synthetic construct
  misc_feature 1..42: note = Staple Strand
  source 1..42: mol_type = other DNA
catcgccattactgaggcttgcagggagttaagccgtcaata

SEQ ID NO: 103 - DNA, 42 bp, synthetic construct
  misc_feature 1..42: note = Staple Strand
  source 1..42: mol_type = other DNA
atattcggtcgaaaataccgaacgaaccaccaggctggctga

SEQ ID NO: 104 - DNA, 42 bp, synthetic construct
  misc_feature 1..42: note = Staple Strand
  source 1..42: mol_type = other DNA
ccttcatcaagtatccagaacaatattaccgccataaccgat

SEQ ID NO: 105 - DNA, 42 bp, synthetic construct
  misc_feature 1..42: note = Staple Strand
  source 1..42: mol_type = other DNA
tctttaggagcggtgtacagaccaggcgcatagcagaagata

SEQ ID NO: 106 - DNA, 42 bp, synthetic construct
  misc_feature 1..42: note = Staple Strand
  source 1..42: mol_type = other DNA
aaacagaggtggctcattcagtgaataaggctatctaaaata

SEQ ID NO: 107 - DNA, 42 bp, synthetic construct
  misc_feature 1..42: note = Staple Strand
  source 1..42: mol_type = other DNA
gtaacaaagctaggcggtcagtattaacaccgtgcggaatcg

SEQ ID NO: 108 - DNA, 42 bp, synthetic construct
  misc_feature 1..42: note = Staple Strand
  source 1..42: mol_type = other DNA
tcataaatattttgcctgagtagaagaactcaccaaatcaac

SEQ ID NO: 109 - DNA, 42 bp, synthetic construct
  misc_feature 1..42: note = Staple Strand
  source 1..42: mol_type = other DNA
aaatcaacagtagactggatagcgtccaataccctgcaacag

SEQ ID NO: 110 - DNA, 42 bp, synthetic construct
  misc_feature 1..42: note = Staple Strand
  source 1..42: mol_type = other DNA
tgccacgctgaaatcaaaaatcaggtctttacgtcagttggc

SEQ ID NO: 111 - DNA, 42 bp, synthetic construct
  misc_feature 1..42: note = Staple Strand
  source 1..42: mol_type = other DNA
gaatgaccatagagccagcagcaaatgaaaaatggcatcaat

SEQ ID NO: 112 - DNA, 42 bp, synthetic construct
  misc_feature 1..42: note = Staple Strand
  source 1..42: mol_type = other DNA
tctactaatagtaaccgttgtagcaatacttccagaaaacga

SEQ ID NO: 113 - DNA, 42 bp, synthetic construct
  misc_feature 1..42: note = Staple Strand
  source 1..42: mol_type = other DNA
tatattttcatcaaaccctcaatcaatatctgcctgactatt

SEQ ID NO: 114 - DNA, 42 bp, synthetic construct
  misc_feature 1..42: note = Staple Strand
  source 1..42: mol_type = other DNA
atagtcagaagaatatacagtaacagtaccttcctgtttagc

SEQ ID NO: 115 - DNA, 42 bp, synthetic construct
  misc_feature 1..42: note = Staple Strand
  source 1..42: mol_type = other DNA
gtaaaatgttttgaaaggaattgaggaaggtttgccctgacg

SEQ ID NO: 116 - DNA, 42 bp, synthetic construct
  misc_feature 1..42: note = Staple Strand
  source 1..42: mol_type = other DNA
agaaacaccagaaataaagaaattgcgtagatgggggtaata

SEQ ID NO: 117 - DNA, 42 bp, synthetic construct
  misc_feature 1..42: note = Staple Strand
  source 1..42: mol_type = other DNA
atgatgaaacaaagggaaccgaactgaccaacaattatttgc

SEQ ID NO: 118 - DNA, 42 bp, synthetic construct
  misc_feature 1..42: note = Staple Strand
  source 1..42: mol_type = other DNA
acgtaaaacagaacgagtagtaaattgggcttgcaaaagaag

SEQ ID NO: 119 - DNA, 42 bp, synthetic construct
  misc_feature 1..42: note = Staple Strand
  source 1..42: mol_type = other DNA
gcagaggcgaatgcaaaagaagttttgccagatttcaggttt

SEQ ID NO: 120 - DNA, 42 bp, synthetic construct
  misc_feature 1..42: note = Staple Strand
  source 1..42: mol_type = other DNA
aacgtcagatgcaaagcggattgcatcaaaaaacaaaatcgc

SEQ ID NO: 121 - DNA, 42 bp, synthetic construct
  misc_feature 1..42: note = Staple Strand
  source 1..42: mol_type = other DNA
cctcccgacttgcgggaggttctgcattaatgaatcggccaa

SEQ ID NO: 122 - DNA, 42 bp, synthetic construct
  misc_feature 1..42: note = Staple Strand
  source 1..42: mol_type = other DNA
taactcacattaattgcgttgagaattaactgaacaccctga

SEQ ID NO: 123 - DNA, 42 bp, synthetic construct
  misc_feature 1..42: note = Staple Strand
  source 1..42: mol_type = other DNA
aaaatgaaaatagcagcctttttaaatttttgttaaatcagc

SEQ ID NO: 124 - DNA, 42 bp, synthetic construct
  misc_feature 1..42: note = Staple Strand
  source 1..42: mol_type = other DNA
aacaggaagattgtataagcatacaattttatcctgaatctt

SEQ ID NO: 125 - DNA, 42 bp, synthetic construct
  misc_feature 1..42: note = Staple Strand
  source 1..42: mol_type = other DNA
agttgctattttgcacccagcaatatttaaattgtaaacgtt

SEQ ID NO: 126 - DNA, 42 bp, synthetic construct
  misc_feature 1..42: note = Staple Strand
  source 1..42: mol_type = other DNA
aatattttgttaaaattcgcaacagagagaataacataaaaa

SEQ ID NO: 127 - DNA, 42 bp, synthetic construct
  misc_feature 1..42: note = Staple Strand
  source 1..42: mol_type = other DNA
cagggaagcgcattagacgggcgctcactgcccgctttccag

SEQ ID NO: 128 - DNA, 42 bp, synthetic construct
  misc_feature 1..42: note = Staple Strand
  source 1..42: mol_type = other DNA
tcgggaaacctgtcgtgccagttgaagccttaaatcaagatt

SEQ ID NO: 129 - DNA, 42 bp, synthetic construct
  misc_feature 1..42: note = Staple Strand
  source 1..42: mol_type = other DNA
accaacgctaacgagcgtctttgtcaatcatatgtaccccgg

SEQ ID NO: 130 - DNA, 42 bp, synthetic construct
  misc_feature 1..42: note = Staple Strand
  source 1..42: mol_type = other DNA
ggtcattgcctgagagtctggacgattttttgtttaacgtca

SEQ ID NO: 131 - DNA, 42 bp, synthetic construct
  misc_feature 1..42: note = Staple Strand
  source 1..42: mol_type = other DNA
ttatcccaatccaaataagaaagcaaacaagagaatcgatga

SEQ ID NO: 132 - DNA, 42 bp, synthetic construct
  misc_feature 1..42: note = Staple Strand
  source 1..42: mol_type = other DNA
acggtaatcgtaaaactagcatccagagcctaatttgccagt

SEQ ID NO: 133 - DNA, 42 bp, synthetic construct
  misc_feature 1..42: note = Staple Strand
  source 1..42: mol_type = other DNA
ccgccaccctcagagccaccatttcatcaacattaaatgtga

SEQ ID NO: 134 - DNA, 42 bp, synthetic construct
  misc_feature 1..42: note = Staple Strand
  source 1..42: mol_type = other DNA
tcattttttaaccaataggaagtagcgcgttttcatcggcat

SEQ ID NO: 135 - DNA, 42 bp, synthetic construct
  misc_feature 1..42: note = Staple Strand
  source 1..42: mol_type = other DNA
aaccatcgatagcagcaccgttggggtgcctaatgagtgagc

SEQ ID NO: 136 - DNA, 42 bp, synthetic construct
  misc_feature 1..42: note = Staple Strand
  source 1..42: mol_type = other DNA
agcttgcatgcctgcaggtcgtagttgcgccgacaatgacaa

SEQ ID NO: 137 - DNA, 42 bp, synthetic construct
  misc_feature 1..42: note = Staple Strand
  source 1..42: mol_type = other DNA
tttcggtcatagcccccttatagagatctacaaaggctatca

SEQ ID NO: 138 - DNA, 42 bp, synthetic construct
  misc_feature 1..42: note = Staple Strand
  source 1..42: mol_type = other DNA
cctcatatattttaaatgcaaaaaaaaggctccaaaaggagc

SEQ ID NO: 139 - DNA, 42 bp, synthetic construct
  misc_feature 1..42: note = Staple Strand
  source 1..42: mol_type = other DNA
tttcacgttgaaaatctccaatgcctgagtaatgtgtaggta

SEQ ID NO: 140 - DNA, 42 bp, synthetic construct
  misc_feature 1..42: note = Staple Strand
  source 1..42: mol_type = other DNA
aagattcaaaagggtgagaaatgagaatagaaaggaacaact

SEQ ID NO: 141 - DNA, 42 bp, synthetic construct
  misc_feature 1..42: note = Staple Strand
  source 1..42: mol_type = other DNA
tcatagttagcgtaacgatcttggtcatagctgtttcctgtg

SEQ ID NO: 142 - DNA, 42 bp, synthetic construct
  misc_feature 1..42: note = Staple Strand
  source 1..42: mol_type = other DNA
ccgagctcgaattcgtaatcaaaagttttgtcgtctttccag

SEQ ID NO: 143 - DNA, 42 bp, synthetic construct
  misc_feature 1..42: note = Staple Strand
  source 1..42: mol_type = other DNA
acgttagtaaatgaattttcttctccgtgggaacaaacggcg

SEQ ID NO: 144 - DNA, 42 bp, synthetic construct
  misc_feature 1..42: note = Staple Strand
  source 1..42: mol_type = other DNA
gcgagtaacaacccgtcggatgtatgggattttgctaaacaa

SEQ ID NO: 145 - DNA, 42 bp, synthetic construct
  misc_feature 1..42: note = Staple Strand
  source 1..42: mol_type = other DNA
ctttaattgtatcggtttatctcacgttggtgtagatgggcg

SEQ ID NO: 146 - DNA, 42 bp, synthetic construct
  misc_feature 1..42: note = Staple Strand
  source 1..42: mol_type = other DNA
gattgaccgtaatgggataggagcttgctttcgaggtgaatt

SEQ ID NO: 147 - DNA, 42 bp, synthetic construct
  misc_feature 1..42: note = Staple Strand
  source 1..42: mol_type = other DNA
ctttcaacagtttcagcggagggccggagacagtcaaatcac

SEQ ID NO: 148 - DNA, 42 bp, synthetic construct
  misc_feature 1..42: note = Staple Strand
  source 1..42: mol_type = other DNA
catcaatatgatattcaaccgtcagagccgccaccctcagaa

SEQ ID NO: 149 - DNA, 42 bp, synthetic construct
  misc_feature 1..42: note = Staple Strand
  source 1..42: mol_type = other DNA
ccaccaccggaaccgcctcccttctagctgataaattaatgc

SEQ ID NO: 150 - DNA, 42 bp, synthetic construct
  misc_feature 1..42: note = Staple Strand
  source 1..42: mol_type = other DNA
tgaaattgttatccgctcacagcattgacaggaggttgaggc

SEQ ID NO: 151 - DNA, 42 bp, synthetic construct
  misc_feature 1..42: note = Staple Strand
  source 1..42: mol_type = other DNA
ccaccaccagagccgccgccaattccacacaacatacgagcc

SEQ ID NO: 152 - DNA, 42 bp, synthetic construct
  misc_feature 1..42: note = Staple Strand
  source 1..42: mol_type = other DNA
tctggccttcctgtagccagcccctcagagccgccaccagaa

SEQ ID NO: 153 - DNA, 42 bp, synthetic construct
  misc_feature 1..42: note = Staple Strand
  source 1..42: mol_type = other DNA
cggagagggtagctatttttgtagcgtttgccatcttttcat

SEQ ID NO: 154 - DNA, 42 bp, synthetic construct
  misc_feature 1..42: note = Staple Strand
  source 1..42: mol_type = other DNA
tcttaaacagcttgataccgaactctagaggatccccgggta

SEQ ID NO: 155 - DNA, 42 bp, synthetic construct
  misc_feature 1..42: note = Staple Strand
  source 1..42: mol_type = other DNA
ggaagcataaagtgtaaagccaatcagtagcgacagaatcaa

SEQ ID NO: 156 - DNA, 42 bp, synthetic construct
  misc_feature 1..42: note = Staple Strand
  source 1..42: mol_type = other DNA
gtttgcctttagcgtcagactcgccatcaaaaataattcgcg

SEQ ID NO: 157 - DNA, 42 bp, synthetic construct
  misc_feature 1..42: note = Staple Strand
  source 1..42: mol_type = other DNA
acaggtagaaagattcatcagactccagccagctttccggca

SEQ ID NO: 158 - DNA, 42 bp, synthetic construct
  misc_feature 1..42: note = Staple Strand
  source 1..42: mol_type = other DNA
catcgtaaccgtgcatctgcctggtttaatttcaactttaat

SEQ ID NO: 159 - DNA, 42 bp, synthetic construct
  misc_feature 1..42: note = Staple Strand
  source 1..42: mol_type = other DNA
attcagtgaataaggcttgccgtaaaacgacggccagtgcca

SEQ ID NO: 160 - DNA, 42 bp, synthetic construct
  misc_feature 1..42: note = Staple Strand
  source 1..42: mol_type = other DNA
cattgtgaattaccttatgcgaaggataaaaatttttagaac

SEQ ID NO: 161 - DNA, 42 bp, synthetic construct
  misc_feature 1..42: note = Staple Strand
  source 1..42: mol_type = other DNA
tagcaaaattaagcaataaagtctactaatagtagtagcatt

SEQ ID NO: 162 - DNA, 42 bp, synthetic construct
  misc_feature 1..42: note = Staple Strand
  source 1..42: mol_type = other DNA
cgaacgagtagatttagtttgcgctattacgccagctggcga

SEQ ID NO: 163 - DNA, 42 bp, synthetic construct
  misc_feature 1..42: note = Staple Strand
  source 1..42: mol_type = other DNA
ggcgatcggtgcgggcctcttaccattagatacatttcgcaa

SEQ ID NO: 164 - DNA, 42 bp, synthetic construct
  misc_feature 1..42: note = Staple Strand
  source 1..42: mol_type = other DNA
atggtcaataacctgtttagcaggcaaagcgccattcgccat

SEQ ID NO: 165 - DNA, 42 bp, synthetic construct
  misc_feature 1..42: note = Staple Strand
  source 1..42: mol_type = other DNA
ccgcttctggtgccggaaacctatattttcatttggggcgcg

SEQ ID NO: 166 - DNA, 42 bp, synthetic construct
  misc_feature 1..42: note = Staple Strand
  source 1..42: mol_type = other DNA
agctgaaaaggtggcatcaatcctcagagcataaagctaaat

SEQ ID NO: 167 - DNA, 42 bp, synthetic construct
  misc_feature 1..42: note = Staple Strand
  source 1..42: mol_type = other DNA
cggttgtaccaaaaacattataactaacggaacaacattatt

SEQ ID NO: 168 - DNA, 42 bp, synthetic construct
  misc_feature 1..42: note = Staple Strand
  source 1..42: mol_type = other DNA
aaaatctacgttaataaaacggaccctgtaatacttttgcgg

SEQ ID NO: 169 - DNA, 42 bp, synthetic construct
  misc_feature 1..42: note = Staple Strand
  source 1..42: mol_type = other DNA
aagggggatgtgctgcaaggcacgccaaaaggaattacgagg

SEQ ID NO: 170 - DNA, 42 bp, synthetic construct
  misc_feature 1..42: note = Staple Strand
  source 1..42: mol_type = other DNA
ttcaactaatgcagatacatagattaagttgggtaacgccag

SEQ ID NO: 171 - DNA, 42 bp, synthetic construct
  misc_feature 1..42: note = Staple Strand
  source 1..42: mol_type = other DNA
tatcggcctcaggaagatcgcttgagatttaggaataccaca

SEQ ID NO: 172 - DNA, 42 bp, synthetic construct
  misc_feature 1..42: note = Staple Strand
  source 1..42: mol_type = other DNA
gagaagcctttatttcaacgcattttaagaactggctcatta

SEQ ID NO: 173 - DNA, 42 bp, synthetic construct
  misc_feature 1..42: note = Staple Strand
  source 1..42: mol_type = other DNA
ggttttcccagtcacgacgttctgacgagaaacaccagaacg

SEQ ID NO: 174 - DNA, 42 bp, synthetic construct
  misc_feature 1..42: note = Staple Strand
  source 1..42: mol_type = other DNA
agtagtaaattgggcttgagaagtttgaggggacgacgacag

SEQ ID NO: 175 - DNA, 42 bp, synthetic construct
  misc_feature 1..42: note = Staple Strand
  source 1..42: mol_type = other DNA
tctttccttatcattccaagacgtaaaacagaaataaagaaa

SEQ ID NO: 176 - DNA, 42 bp, synthetic construct
  misc_feature 1..42: note = Staple Strand
  source 1..42: mol_type = other DNA
ttgtttggattatacttctgaaaagttaccagaaggaaaccg

SEQ ID NO: 177 - DNA, 42 bp, synthetic construct
  misc_feature 1..42: note = Staple Strand
  source 1..42: mol_type = other DNA
aatgaaatagcaatagctatcaatggattatttacattggca

SEQ ID NO: 178 - DNA, 42 bp, synthetic construct
  misc_feature 1..42: note = Staple Strand
  source 1..42: mol_type = other DNA
ccagccattgcaacaggaaaagccgtttttattttcatcgta

SEQ ID NO: 179 - DNA, 42 bp, synthetic construct
  misc_feature 1..42: note = Staple Strand
  source 1..42: mol_type = other DNA
gcactcatcgagaacaagcaaacgctcatggaaatacctaca

SEQ ID NO: 180 - DNA, 42 bp, synthetic construct
  misc_feature 1..42: note = Staple Strand
  source 1..42: mol_type = other DNA
ttttgacgctcaatcgtctgattaccgaagccctttttaaga

SEQ ID NO: 181 - DNA, 42 bp, synthetic construct
  misc_feature 1..42: note = Staple Strand
  source 1..42: mol_type = other DNA
aaagtaagcagatagccgaacataatggaagggttagaacct

SEQ ID NO: 182 - DNA, 42 bp, synthetic construct
  misc_feature 1..42: note = Staple Strand
  source 1..42: mol_type = other DNA
accatatcaaaattatttgcaacgggtattaaaccaagtacc

SEQ ID NO: 183 - DNA, 42 bp, synthetic construct
  misc_feature 1..42: note = Staple Strand
  source 1..42: mol_type = other DNA
ggaatcattaccgcgcccaattcaaactatcggccttgctgg

SEQ ID NO: 184 - DNA, 42 bp, synthetic construct
  misc_feature 1..42: note = Staple Strand
  source 1..42: mol_type = other DNA
aattaaccgttgtagcaatacccaataataagagcaagaaac

SEQ ID NO: 185 - DNA, 42 bp, synthetic construct
  misc_feature 1..42: note = Staple Strand
  source 1..42: mol_type = other DNA
acaaagtcagagggtaattgaccgcctggccctgagagagtt

SEQ ID NO: 186 - DNA, 42 bp, synthetic construct
  misc_feature 1..42: note = Staple Strand
  source 1..42: mol_type = other DNA
tattgggcgccagggtggtttaacgcgaggcgttttagcgaa

SEQ ID NO: 187 - DNA, 42 bp, synthetic construct
  misc_feature 1..42: note = Staple Strand
  source 1..42: mol_type = other DNA
aggcttatccggtattctaagttcttttcaccagtgagacgg

SEQ ID NO: 188 - DNA, 42 bp, synthetic construct
  misc_feature 1..42: note = Staple Strand
  source 1..42: mol_type = other DNA
gcaacagctgattgcccttcagcgctaatatcagagagataa

SEQ ID NO: 189 - DNA, 42 bp, synthetic construct
  misc_feature 1..42: note = Staple Strand
  source 1..42: mol_type = other DNA
cccacaagaattgagttaagcttctttgattagtaataacat

SEQ ID NO: 190 - DNA, 42 bp, synthetic construct
  misc_feature 1..42: note = Staple Strand
  source 1..42: mol_type = other DNA
cacttgcctgagtagaagaacagcaagcaaatcagatataga

SEQ ID NO: 191 - DNA, 42 bp, synthetic construct
  misc_feature 1..42: note = Staple Strand
  source 1..42: mol_type = other DNA
ttccagtaagcgtcatacatgtgacctgaaagcgtaagaata

SEQ ID NO: 192 - DNA, 42 bp, synthetic construct
  misc_feature 1..42: note = Staple Strand
  source 1..42: mol_type = other DNA
gattcaccagtcacacgaccaaaggtgaattatcaccgtcac

SEQ ID NO: 193 - DNA, 42 bp, synthetic construct
  misc_feature 1..42: note = Staple Strand
  source 1..42: mol_type = other DNA
caaaagggcgacattcaaccgaattcatcaatataatcctga

SEQ ID NO: 194 - DNA, 42 bp, synthetic construct
  misc_feature 1..42: note = Staple Strand
  source 1..42: mol_type = other DNA
tttacaaacaattcgacaactactttttcatgaggaagtttc

SEQ ID NO: 195 - DNA, 42 bp, synthetic construct
  misc_feature 1..42: note = Staple Strand
  source 1..42: mol_type = other DNA
caaccatcgcccacgcataacaaagaacgtggactccaacgt

SEQ ID NO: 196 - DNA, 42 bp, synthetic construct
  misc_feature 1..42: note = Staple Strand
  source 1..42: mol_type = other DNA
gcagcaagcggtccacgctggggccggaaacgtcaccaatga

SEQ ID NO: 197 - DNA, 42 bp, synthetic construct
  misc_feature 1..42: note = Staple Strand
  source 1..42: mol_type = other DNA
cgacttgagccatttgggaataaagagtctgtccatcacgca

SEQ ID NO: 198 - DNA, 42 bp, synthetic construct
  misc_feature 1..42: note = Staple Strand
  source 1..42: mol_type = other DNA
tggttgctttgacgagcacgtcttttgcgggatcgtcaccct

SEQ ID NO: 199 - DNA, 42 bp, synthetic construct
  misc_feature 1..42: note = Staple Strand
  source 1..42: mol_type = other DNA
cttgcagggagttaaaggccgataacgtgctttcctcgttag

SEQ ID NO: 200 - DNA, 42 bp, synthetic construct
  misc_feature 1..42: note = Staple Strand
  source 1..42: mol_type = other DNA
aatcagagcgggagctaaacaccgtaacactgagtttcgtca

SEQ ID NO: 201 - DNA, 42 bp, synthetic construct
  misc_feature 1..42: note = Staple Strand
  source 1..42: mol_type = other DNA
ggaggtttagtaccgccaccctgagtaacattatcattttgc

SEQ ID NO: 202 - DNA, 42 bp, synthetic construct
  misc_feature 1..42: note = Staple Strand
  source 1..42: mol_type = other DNA
acgttattaattttaaaagtttcagaaccgccaccctcagaa

SEQ ID NO: 203 - DNA, 42 bp, synthetic construct
  misc_feature 1..42: note = Staple Strand
  source 1..42: mol_type = other DNA
ccgccaccctcagagccaccagaatggctattagtctttaat

SEQ ID NO: 204 - DNA, 42 bp, synthetic construct
  misc_feature 1..42: note = Staple Strand
  source 1..42: mol_type = other DNA
cgtggcacagacaatatttttccctcattttcagggatagca

SEQ ID NO: 205 - DNA, 42 bp, synthetic construct
  misc_feature 1..42: note = Staple Strand
  source 1..42: mol_type = other DNA
cagcagcgaaagacagcatcgacatcgccattaaaaataccg

SEQ ID NO: 206 - DNA, 42 bp, synthetic construct
  misc_feature 1..42: note = Staple Strand
  source 1..42: mol_type = other DNA
gcgcgaactgatagccctaaagaacgagggtagcaacggcta

SEQ ID NO: 207 - DNA, 42 bp, synthetic construct
  misc_feature 1..42: note = Staple Strand
  source 1..42: mol_type = other DNA
agcccaataggaacccatgtaggaggccgattaaagggattt

SEQ ID NO: 208 - DNA, 42 bp, synthetic construct
  misc_feature 1..42: note = Staple Strand
  source 1..42: mol_type = other DNA
atcaaaagaatagcccgagatgtagcattccacagacagccc

SEQ ID NO: 209 - DNA, 42 bp, synthetic construct
  misc_feature 1..42: note = Staple Strand
  source 1..42: mol_type = other DNA
ccagtacaaactacaacgcctagggttgagtgttgttccagt

SEQ ID NO: 210 - DNA, 42 bp, synthetic construct
  misc_feature 1..42: note = Staple Strand
  source 1..42: mol_type = other DNA
tagacaggaacggtacgccaggcgcagtctctgaatttaccg

SEQ ID NO: 211 - DNA, 42 bp, synthetic construct
  misc_feature 1..42: note = Staple Strand
  source 1..42: mol_type = other DNA
aggtcagacgattggccttgaaatcggcaaaatcccttataa

SEQ ID NO: 212 - DNA, 42 bp, synthetic construct
  misc_feature 1..42: note = Staple Strand
  source 1..42: mol_type = other DNA
cctgtttgatggtggttccgatattcacaaacaaataaatcc

SEQ ID NO: 213 - DNA, 42 bp, synthetic construct
  misc_feature 1..42: note = Staple Strand
  source 1..42: mol_type = other DNA
tcattaaagccagaatggaaaaatcctgagaagtgtttttat

SEQ ID NO: 214 - DNA, 42 bp, synthetic construct
  misc_feature 1..42: note = Staple Strand
  source 1..42: mol_type = other DNA
ggaacaaagaaaccaccagaagggtcagtgccttgagtaaca

SEQ ID NO: 215 - DNA, 42 bp, synthetic construct
  misc_feature 1..42: note = Staple Strand
  source 1..42: mol_type = other DNA
tactggtaataagttttaacgggagcggaattatcatcatat

SEQ ID NO: 216 - DNA, 42 bp, synthetic construct
  misc_feature 1..42: note = Staple Strand
  source 1..42: mol_type = other DNA
ccaacagagatagaacccttcgcttttgatgatacaggagtg

SEQ ID NO: 217 - DNA, 42 bp, synthetic construct
  misc_feature 1..42: note = Staple Strand
  source 1..42: mol_type = other DNA
aatcagtgaggccaccgagtatagagccagcaaaatcaccag

SEQ ID NO: 218 - DNA, 42 bp, synthetic construct
  misc_feature 1..42: note = Staple Strand
  source 1..42: mol_type = other DNA
tagcaccattaccattagcaatttgccccagcaggcgaaaat

SEQ ID NO: 219 - DNA, 42 bp, synthetic construct
  misc_feature 1..42: note = Staple Strand
  source 1..42: mol_type = other DNA
ttggaacaagagtccactattcgatatattcggtcgctgagg

SEQ ID NO: 220 - DNA, 42 bp, synthetic construct
  misc_feature 1..42: note = Staple Strand
  source 1..42: mol_type = other DNA
cagaggctttgaggactaaagcgtattaaatcctttgcccga

SEQ ID NO: 221 - DNA, 42 bp, synthetic construct
  misc_feature 1..42: note = Staple Strand
  source 1..42: mol_type = other DNA
tcctgattatcagatgatggcattgagggagggaaggtaaat

SEQ ID NO: 222 - DNA, 42 bp, synthetic construct
  misc_feature 1..42: note = Staple Strand
  source 1..42: mol_type = other DNA
attgacggaaattattcattagtaataaaagggacattctgg

SEQ ID NO: 223 - DNA, 42 bp, synthetic construct
  misc_feature 1..42: note = Staple Strand
  source 1..42: mol_type = other DNA
tttgccagagggggtaatagtgtgccacgctgagagccagca

SEQ ID NO: 224 - DNA, 42 bp, synthetic construct
  misc_feature 1..42: note = Staple Strand
  source 1..42: mol_type = other DNA
aacgaaccaccagcagaagatatgaacggtgtacagaccagg

SEQ ID NO: 225 - DNA, 42 bp, synthetic construct
  misc_feature 1..42: note = Staple Strand
  source 1..42: mol_type = other DNA
cggaacgaggcgcagacggtcgaggatttagaagtattagac

SEQ ID NO: 226 - DNA, 42 bp, synthetic construct
  misc_feature 1..42: note = Staple Strand
  source 1..42: mol_type = other DNA
caaagggcgaaaaaccgtctaatcaacgtaacaaagctgctc

SEQ ID NO: 227 - DNA, 42 bp, synthetic construct
  misc_feature 1..42: note = Staple Strand
  source 1..42: mol_type = other DNA
cgcataggctggctgaccttcgccgctacagggcgcgtacta

SEQ ID NO: 228 - DNA, 42 bp, synthetic construct
  misc_feature 1..42: note = Staple Strand
  source 1..42: mol_type = other DNA
cgtggcgagaaaggaagggaaatatgcaactaaagtacggtg

SEQ ID NO: 229 - DNA, 42 bp, synthetic construct
  misc_feature 1..42: note = Staple Strand
  source 1..42: mol_type = other DNA
aggattagagagtacctttaagaaaggaattgaggaaggtta

SEQ ID NO: 230 - DNA, 42 bp, synthetic construct
  misc_feature 1..42: note = Staple Strand
  source 1..42: mol_type = other DNA
tcagttggcaaatcaacagttttgctccttttgataagaggt

SEQ ID NO: 231 - DNA, 42 bp, synthetic construct
  misc_feature 1..42: note = Staple Strand
  source 1..42: mol_type = other DNA
catttttgcggatggcttagatcaccttgctgaacctcaaat

SEQ ID NO: 232 - DNA, 42 bp, synthetic construct
  misc_feature 1..42: note = Staple Strand
  source 1..42: mol_type = other DNA
gcaaatgaaaaatctaaagcagcttaattgctgaatataatg

SEQ ID NO: 233 - DNA, 42 bp, synthetic construct
  misc_feature 1..42: note = Staple Strand
  source 1..42: mol_type = other DNA
ctgtagctcaacatgttttaagaaagcgaaaggagcgggcgc

SEQ ID NO: 234 - DNA, 42 bp, synthetic construct
  misc_feature 1..42: note = Staple Strand
  source 1..42: mol_type = other DNA
taaagcactaaatcggaacccaacagttgattcccaattctg

SEQ ID NO: 235 - DNA, 42 bp, synthetic construct
  misc_feature 1..42: note = Staple Strand
  source 1..42: mol_type = other DNA
tctggaagtttcattccatattaaagggagcccccgatttag

SEQ ID NO: 236 - DNA, 42 bp, synthetic construct
  misc_feature 1..42: note = Staple Strand
  source 1..42: mol_type = other DNA
tagggcgctggcaagtgtagcagaggcttttgcaaaagaagt

SEQ ID NO: 237 - DNA, 42 bp, synthetic construct
  misc_feature 1..42: note = Staple Strand
  source 1..42: mol_type = other DNA
catagtaagagcaacactatcttttttggggtcgaggtgccg

SEQ ID NO: 238 - DNA, 42 bp, synthetic construct
  misc_feature 1..42: note = Staple Strand
  source 1..42: mol_type = other DNA
tgaaccatcacccaaatcaagataaccctcgtttaccagacg

SEQ ID NO: 239 - DNA, 42 bp, synthetic construct
  misc_feature 1..42: note = Staple Strand
  source 1..42: mol_type = other DNA
acgataaaaaccaaaatagcgggtcacgctgcgcgtaaccac

SEQ ID NO: 240 - DNA, 42 bp, synthetic construct
  misc_feature 1..42: note = Staple Strand
  source 1..42: mol_type = other DNA
tctaaaatatctttaggagcaataaatattcattgaatcccc

SEQ ID NO: 241 - DNA, 42 bp, synthetic construct
  misc_feature 1..42: note = Staple Strand
  source 1..42: mol_type = other DNA
gtccaatactgcggaatcgtcctaacaactaatagattagag

SEQ ID NO: 242 - DNA, 42 bp, synthetic construct
  misc_feature 1..42: note = Staple Strand
  source 1..42: mol_type = other DNA
gtattaacaccgcctgcaacaaaaatgtttagactggatagc

SEQ ID NO: 243 - DNA, 42 bp, synthetic construct
  misc_feature 1..42: note = Staple Strand
  source 1..42: mol_type = other DNA
cacacccgccgcgcttaatgcatcaagagtaatcttgacaag

SEQ ID NO: 244 - DNA, 42 bp, synthetic construct
  misc_feature 1..42: note = Staple Strand
  source 1..42: mol_type = other DNA
aaccggatattcattacccaatcagggcgatggcccactacg

SEQ ID NO: 245 - DNA, 42 bp, synthetic construct
  misc_feature 1..42: note = Staple Strand
  source 1..42: mol_type = other DNA
ccgtcaatagataatacatttaatcataagggaaccgaactg

SEQ ID NO: 246 - DNA, 42 bp, synthetic construct
  misc_feature 1..42: note = Staple Strand
  source 1..42: mol_type = other DNA
accaactttgaaagaggacagaaaacagaggtgaggcggtca

SEQ ID NO: 247 - DNA, 42 bp, synthetic construct
  misc_feature 1..42: note = Staple Strand
  source 1..42: mol_type = other DNA
atcaacaatagataagtcctgtgtccagacgacgacaataaa

SEQ ID NO: 248 - DNA, 42 bp, synthetic construct
  misc_feature 1..42: note = Staple Strand
  source 1..42: mol_type = other DNA
gcagaggcattttcgagccaggtatgttagcaaacgtagaaa

SEQ ID NO: 249 - DNA, 42 bp, synthetic construct
  misc_feature 1..42: note = Staple Strand
  source 1..42: mol_type = other DNA
aggaaacgcaataataacggattgctttgaataccaagttac

SEQ ID NO: 250 - DNA, 42 bp, synthetic construct
  misc_feature 1..42: note = Staple Strand
  source 1..42: mol_type = other DNA
gtcagatgaatatacagtaacaaaccaatcaataatcggctg

SEQ ID NO: 251 - DNA, 42 bp, synthetic construct
  misc_feature 1..42: note = Staple Strand
  source 1..42: mol_type = other DNA
tcctaatttacgagcatgtagagtaccttttacatcgggaga

SEQ ID NO: 252 - DNA, 42 bp, synthetic construct
  misc_feature 1..42: note = Staple Strand
  source 1..42: mol_type = other DNA
aacaataacggattcgcctgaatacccaaaagaactggcatg

SEQ ID NO: 253 - DNA, 42 bp, synthetic construct
  misc_feature 1..42: note = Staple Strand
  source 1..42: mol_type = other DNA
attaagactccttattacgcataataagagaatataaagtac

SEQ ID NO: 254 - DNA, 42 bp, synthetic construct
  misc_feature 1..42: note = Staple Strand
  source 1..42: mol_type = other DNA
cgacaaaaggtaaagtaattcaacaagaaaaataatatccca

SEQ ID NO: 255 - DNA, 42 bp, synthetic construct
  misc_feature 1..42: note = Staple Strand
  source 1..42: mol_type = other DNA
cattaaacgggtaaaatacgttgagtgaataaccttgcttct

SEQ ID NO: 256 - DNA, 42 bp, synthetic construct
  misc_feature 1..42: note = Staple Strand
  source 1..42: mol_type = other DNA
aaaatcgcgcagaggcgaattatggtttaccagcgccaaaga

SEQ ID NO: 257 - DNA, 42 bp, synthetic construct
  misc_feature 1..42: note = Staple Strand
  source 1..42: mol_type = other DNA
ataaaagaaacgcaaagacaccaacgccaacatgtaatttag

SEQ ID NO: 258 - DNA, 42 bp, synthetic construct
  misc_feature 1..42: note = Staple Strand
  source 1..42: mol_type = other DNA
gtgataaataaggcgttaaatagaatacactaaaacactcat

SEQ ID NO: 259 - DNA, 42 bp, synthetic construct
  misc_feature 1..42: note = Staple Strand
  source 1..42: mol_type = other DNA
acctaaaacgaaagaggcaaaaagaataaacaccggaatcat

SEQ ID NO: 260 - DNA, 42 bp, synthetic construct
  misc_feature 1..42: note = Staple Strand
  source 1..42: mol_type = other DNA
aattactagaaaaagcctgttggataagtgccgtcgagaggg

SEQ ID NO: 261 - DNA, 42 bp, synthetic construct
  misc_feature 1..42: note = Staple Strand
  source 1..42: mol_type = other DNA
gggttttgctcagtaccaggctagtatcatatgcgttataca

SEQ ID NO: 262 - DNA, 42 bp, synthetic construct
  misc_feature 1..42: note = Staple Strand
  source 1..42: mol_type = other DNA
tacatttaacaatttcatttgataggtgtatcaccgtactca

SEQ ID NO: 263 - DNA, 42 bp, synthetic construct
  misc_feature 1..42: note = Staple Strand
  source 1..42: mol_type = other DNA
ttgatataagtatagcccggaaattaccttttttaatggaaa

SEQ ID NO: 264 - DNA, 42 bp, synthetic construct
  misc_feature 1..42: note = Staple Strand
  source 1..42: mol_type = other DNA
aattcttaccagtataaagccgtattaagaggctgagactcc

SEQ ID NO: 265 - DNA, 42 bp, synthetic construct
  misc_feature 1..42: note = Staple Strand
  source 1..42: mol_type = other DNA
gtgcccgtataaacagttaatcatcaagaaaacaaaattaat

SEQ ID NO: 266 - DNA, 42 bp, synthetic construct
  misc_feature 1..42: note = Staple Strand
  source 1..42: mol_type = other DNA
aaaagaagatgatgaaacaaagccccctgcctatttcggaac

SEQ ID NO: 267 - DNA, 42 bp, synthetic construct
  misc_feature 1..42: note = Staple Strand
  source 1..42: mol_type = other DNA
ctattattctgaaacatgaaaaacgctcaacagtagggctta

SEQ ID NO: 268 - DNA, 42 bp, synthetic construct
  misc_feature 1..42: note = Staple Strand
  source 1..42: mol_type = other DNA
attgagaatcgccatatttaacacggaataagtttattttgt

SEQ ID NO: 269 - DNA, 42 bp, synthetic construct
  misc_feature 1..42: note = Staple Strand
  source 1..42: mol_type = other DNA
cacaatcaatagaaaattcatattcatttcaattacctgagc

SEQ ID NO: 270 - DNA, 42 bp, synthetic construct
  misc_feature 1..42: note = Staple Strand
  source 1..42: mol_type = other DNA
cagtacataaatcaatatatgaatgccactacgaaggcacca

SEQ ID NO: 271 - DNA, 42 bp, synthetic construct
  misc_feature 1..42: note = Staple Strand
  source 1..42: mol_type = other DNA
gtaaatcgtcgctattaattaacctgctccatgttacttagc

SEQ ID NO: 272 - DNA, 42 bp, synthetic construct
  misc_feature 1..42: note = Staple Strand
  source 1..42: mol_type = other DNA
agcgcgaaacaaagtacaacgatggtttgaaataccgaccgt

SEQ ID NO: 273 - DNA, 42 bp, synthetic construct
  misc_feature 1..42: note = Staple Strand
  source 1..42: mol_type = other DNA
tataactatatgtaaatgctgcaaatatcgcgttttaattcg

SEQ ID NO: 274 - DNA, 42 bp, synthetic construct
  misc_feature 1..42: note = Staple Strand
  source 1..42: mol_type = other DNA
aagaggaagcccgaaagacttatgcaaatccaatcgcaagac

SEQ ID NO: 275 - DNA, 42 bp, synthetic construct
  misc_feature 1..42: note = Staple Strand
  source 1..42: mol_type = other DNA
tagtgaatttatcaaaatcatggaagcaaactccaacaggtc

SEQ ID NO: 276 - DNA, 42 bp, synthetic construct
  misc_feature 1..42: note = Staple Strand
  source 1..42: mol_type = other DNA
agcttcaaagcgaaccagaccaggtctgagagactacctttt

SEQ ID NO: 277 - DNA, 42 bp, synthetic construct
  misc_feature 1..42: note = Staple Strand
  source 1..42: mol_type = other DNA
aaagaacgcgagaaaacttttctgactattatagtcagaagc

SEQ ID NO: 278 - DNA, 42 bp, synthetic construct
  misc_feature 1..42: note = Staple Strand
  source 1..42: mol_type = other DNA
ctcaaatgctttaaacagttctaagacgctgagaagagtcaa

SEQ ID NO: 279 - DNA, 42 bp, synthetic construct
  misc_feature 1..42: note = Staple Strand
  source 1..42: mol_type = other DNA
aaacatagcgatagcttagatagaaaacgagaatgaccataa

SEQ ID NO: 280 - DNA, 42 bp, synthetic construct
  misc_feature 1..42: note = Staple Strand
  source 1..42: mol_type = other DNA
atcaaaaatcaggtctttacctcaaatatattttagttaatt

SEQ ID NO: 281 - DNA, 42 bp, synthetic construct
  misc_feature 1..42: note = Staple Strand
  source 1..42: mol_type = other DNA
tcatcttctgacctaaatttagagatttgtatcatcgcctga

SEQ ID NO: 282 - DNA, 42 bp, synthetic construct
  misc_feature 1..42: note = Staple Strand
  source 1..42: mol_type = other DNA
taaattgtgtcgaaatccgcgattttcccttagaatccttga